(19) United States
(12) Patent Application Publication
(10) Pub. No.: US 2003/0018616 A1
(43) Pub. Date: Jan. 23, 2003

(54) Systems, methods and computer program products for integrating databases to create an ontology network

(76) Inventors: John Thompson Wilbanks, Chapel Hill, NC (US); Joshua Lerner Levy, Chapel Hill, NC (US); Suresh Toby Segaran, Chapel Hill, NC (US); Richard N. Gardner, Raleigh, NC (US)

Correspondence Address: MYERS BIGEL SIBLEY & SAJOVEC, PO BOX 37428, RALEIGH, NC, 27627, US

(21) Appl. No.: 10/154,407

(22) Filed: May 23, 2002

Related U.S. Application Data
(60) Provisional application No. 60/296,018, filed on Jun. 5, 2001.
(60) Provisional application No. 60/356,616, filed on Feb. 13, 2002.

(51) Int. Cl. G06F 7/00; G06F 17/30; G06F 19/00; G01N 33/48; G01N 33/50

(52) U.S. Cl. 707/2; 702/19

(57) ABSTRACT

Databases are integrated by obtaining an entity-relationship model for each of the databases, and identifying related entities in the entity-relationship models of at least two of the databases. At least two of the related entities that are identified are linked, to thereby create an entity-relationship model that integrates the plurality of databases. The entity-relationship model that integrates the databases provides an ontology network that integrates the diverse ontologies that are represented by the independent databases. By navigating the entity-relationship model in response to queries, discovery may be obtained that may not be obtainable from any one of the independent databases.

### CROSS REFERENCE TO RELATED APPLICATIONS

This application is related to and claims the benefit of U.S. application Ser. No.______ to Wilbanks, Levy, Segaran and Gardner, filed May 13, 2002, entitled Systems, Methods and Computer Program Products for Integrating Biological/Chemical Databases to Create an Ontology Network (Attorney Docket 9223-10), which itself is related to and claims the benefit of Provisional Application Serial No. 60/296,018 to Levy and Segaran, filed Jun. 5, 2001, entitled Cell: A Cross-Referenced Ontological Database for Biological Data; and Provisional Application Serial No. 60/356,616 to Gardner and Wilbanks, filed Feb. 13, 2002, entitled Ontology Networks, a New Foundation for Discovery, all of which are assigned to the assignee of the present application, the disclosures of all of which are hereby incorporated herein by reference in their entirety as if set forth fully herein.

### FIELD OF THE INVENTION

This invention relates to data processing systems, methods and computer program products, and more particularly to database systems, methods and computer program products.

### BACKGROUND OF THE INVENTION

The manufacturing and service industries, as well as government entities, generate massive amounts of private and public data. Unfortunately, this enormous increase in the amount of data may not lead to corresponding advances in discovery, because the sheer volume of data may outpace the ability of experts to transform that data into knowledge.

The massive volume of data that is being generated also may be accompanied by a large diversity of data sources that may generate the data. For example, public, private, proprietary, governmental and other databases from various data sources may be produced. Unfortunately, it may be difficult to integrate these heterogeneous data sources.

One conventional approach for data integration uses a data warehouse and data mining techniques. A data warehouse may use a relational database and a star model in which searchable database fields are stored in their own tables, forming a star around a table of records. Unfortunately, it may be difficult to integrate new types of data without significant modification to the table structure. Moreover, querying the assembled information using conventional data mining techniques also may present potential problems. These queries may range in sophistication from simple use of Boolean operators, data search engines such as Internet-based search tools, and/or more sophisticated query languages that employ relational inquiries into the database. Unfortunately, these queries may require significant knowledge of the data sources, the structure of the assembled data, and/or experience in the use of query languages. The use of Internet-based search engines may yield inaccurate yet exhaustive reams of information that may not be relevant to the original request.

Another conventional approach that may be used for data integration is the flat-file or link-driven federation, wherein users can perform text searching on the databases independently, and then jump to different databases, for example via World Wide Web links. Although a flat-file or link-driven federation may simplify searching for non-expert users, it may be difficult to search across multiple databases simultaneously. Moreover, it may be difficult to obtain desired information for data records that only are indirectly and/or inferentially linked.

Another conventional integration technique is referred to as a wrapper or view, which can provide cross-database querying without moving data from the original databases. For each database, a separate driver may be designed that can query the database. A wrapper can then ask several databases for some results and bring them together to find intersections. Unfortunately, it may be difficult to bring in new data types, as new drivers may need to be provided for every new data source. Moreover, queries may be slow and memory-intensive, because all relevant databases may need to be queried for their entire result set before elimination by any other parts of the query is performed. Finally, relationships may not be provided unless specified in the queries and/or wrappers.

### SUMMARY OF THE INVENTION

Some embodiments of the present invention integrate a plurality of databases by obtaining an entity-relationship model for each of the plurality of databases, and identifying related entities, including identical entities, in the entity-relationship models of at least two of the databases. At least two of the related entities that are identified are linked, to thereby create an entity-relationship model that integrates the plurality of databases. In some embodiments, when the entities are identical entities, they are merged. In some embodiments, each of the plurality of databases represents an ontology and the entity-relationship model that integrates the plurality of databases creates an ontology network.

Accordingly, ontology networks according to some embodiments of the present invention can link related entities in entity-relationship models of independent databases, to thereby create a single entity-relationship model for the independent databases. By navigating the single entity-relationship model in response to queries, discovery may be obtained that may not be obtainable from any one of the independent databases.

In some embodiments, linking is performed by merging at least two of the identical entities that are identified into a single entity in the entity-relationship model that integrates the plurality of databases. In other embodiments, merging is accomplished by establishing a plurality of aliases for the single entity in the entity-relationship model that integrates the plurality of databases, a respective alias of which refers to a respective one of the identical entities that are identified.

In some embodiments, the traversing is performed from a starting entity to an ending entity in response to a query that specifies the starting entity and the ending entity. In other embodiments, the entities are traversed from a starting entity to a plurality of ending entities in response to a query that specifies the starting entity. In yet other embodiments, the entities are traversed in response to a query and in response to at least one path rule. In some embodiments, the at least one path rule specifies the type of path to use in traversing through the plurality of entities, the type of path not to use in traversing through the plurality of entities, the type of ending entity that can be included in the query results, the type of ending entity that is not to be included in the query results, the type of relationship to be used in traversing through the plurality of entities, the type of relationship that is not to be used in traversing through the plurality of entities and/or a confidence level to be achieved in traversing through the plurality of entities. In still other embodiments, groups of relationships may be classified into a class of relationships, and the at least one path rule can specify a class of relationships to be included or excluded. Multiple classes can be assigned to a given relationship.

In other embodiments, the query results are stored as at least one new relationship in the entity-relationship model that integrates the plurality of databases, to thereby store knowledge that was derived from the query in the entity-relationship model that integrates the plurality of databases. In still other embodiments, a confidence level is assigned to at least one of the relationships in the entity-relationship model that integrates the plurality of databases. In still other embodiments, query results also may be based on assigned confidence levels.

According to other embodiments of the present invention, a new database may be integrated with a plurality of databases, by providing an entity-relationship model of the plurality of database that links at least some related entities in at least two of the databases. An entity-relationship model for the new database is obtained. Related entities in the entity-relationship model of the new database and the entity-relationship model of the plurality of databases are identified. At least two of the related entities that are identified are linked, to thereby create an entity-relationship model that integrates the plurality of databases and the new database. In other embodiments, the entity-relationship model of the plurality of databases that links at least some related entities in the at least two of the databases provides an ontology network and the entity-relationship model of the new database represents an ontology.

In other embodiments of the invention, when linking identical entities, the at least two of the identical entities that are identified are merged into a single entity in the entity-relationship model that integrates the plurality of databases and the new database. In other embodiments, merging may be accomplished by establishing a plurality of aliases for the single entity in the entity-relationship model that integrates the plurality of databases and the new database. A respective alias refers to a respective one of the at least two of the identical entities that are identified.

In other embodiments, the new database is an updated version of one of the plurality of databases. In some of these embodiments, at least one entity is identified that is in the one of the plurality of databases and that has been deleted from the updated version of the one of the plurality of databases. An alias that is associated with the at least one entity is removed. In still other embodiments, at least one entity is split based upon the alias that was removed. In yet other embodiments, an image of the at least one record that has been deleted may be retained in the plurality of databases, so as to allow an archival history to be maintained. In still other embodiments, multiple images or instances of the entity/relationship structure may be maintained to reflect updates and/or deleted records and/or query results, and these multiple instances may be correlated to one another to obtain new knowledge.

In still other embodiments, when adding a new database, entities in the new database that do not correspond to at least one of the entities in the entity-relationship model that integrates the plurality of databases and the new database are identified. At least one new entity is added to the entity-relationship model that corresponds to the entities in the new database that do not correspond to at least one of the entities in the entity-relationship model.

Data processing systems according to some embodiments of the present invention include an ontology network engine that is configured to build an integrated entity-relationship model of a plurality of independent databases. The entity-relationship model comprises a plurality of entities including links and also comprises a plurality of relationships. In some embodiments, a metadata database is configured to store therein the integrated entity-relationship model of the plurality of independent databases. In other embodiments, a loader is configured to load an independent entity-relationship model of each of the independent databases into the ontology network engine. The independent databases may be loaded in a typeless format. Other embodiments include a virtual experiment layer that is configured to conduct virtual experiments on the integrated entity-relationship model. Yet other embodiments include a discovery layer that is configured to discover knowledge from the integrated entity-relationship model. Moreover, in still other embodiments, the integrated entity-relationship model provides a data structure. Finally, it will be understood that any of the embodiments described herein may be provided as systems, methods and/or computer program products.

### BRIEF DESCRIPTION OF THE DRAWINGS

FIGS. 1 and 2 illustrate conceptual overviews of environments in which some embodiments of the present invention may be used.

FIG. 3 is a hardware/software block diagram of some embodiments of the present invention.

FIG. 4 is a software architecture diagram of some embodiments of the present invention.

FIG. 5 is a flowchart of operations for integrating databases according to some embodiments of the present invention.

FIG. 6 is a flowchart of operations for integrating a new database into a plurality of databases according to some embodiments of the present invention.

FIG. 7 is a flowchart of operations for querying a plurality of databases according to some embodiments of the present invention.

FIG. 8 is a flowchart of operations for integrating databases according to some embodiments of the present invention.

FIG. 9 is a flowchart of operations for integrating new databases according to some embodiments of the present invention.

FIG. 10 is a flowchart of operations for performing queries according to some embodiments of the present invention.

FIG. 11 is a block diagram of a data processing architecture that may be used with some embodiments of the present invention.

FIGS. 12A and 12B, which together form FIG. 12, is an entity-relationship diagram of a conceptual schema for an ontology network according to some embodiments of the present invention.

FIGS. 13 and 14 are flowcharts of operations for integrating databases and integrating new databases according to some embodiments of the present invention.

FIG. 15 is a flowchart illustrating operations for traversing an ontology network using path rules according to some embodiments of the present invention.

FIGS. 16 and 17 are flowcharts of operations for querying an ontology network according to some embodiments of the present invention.

FIG. 18 illustrates a conceptual overview of environments in which some embodiments of the present invention may be used.

FIGS. 19 and 20 illustrate examples of ontology networks that can be used to link personal data, securities data and government data according to some embodiments of the present invention.

### DETAILED DESCRIPTION OF PREFERRED EMBODIMENTS

The present invention now will be described more fully hereinafter with reference to the accompanying figures, in which embodiments of the invention are shown. This invention may, however, be embodied in many alternate forms and should not be construed as limited to the embodiments set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like numbers refer to like elements throughout the description of the figures.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

Definitions

As used herein, the following terms have the following meanings:

Entity-relationship: A data model that views information as a set of basic objects (entities) and relationships among these entities. An entity is an object or concept about which information is stored. An entity may have attributes which are the properties or characteristics of the entity. Relationships indicate how two entities share information. Relationships may also have attributes or properties. The entity-relationship model was originally developed by Dr. Peter P. Chen and was adopted as the meta model for the American National Standards Institute (ANSI) Standard on Information Resource Directory System (IRDS).

Ontology: A structured vocabulary of terms and some specification of their meaning and/or relationships among one another based on a set of beliefs about the terms and their meanings/relationships. The structure can be explicit and/or implicit.

Other terms used herein have their ordinary meaning to those having skill in the art, unless specified otherwise, and, therefore, need not be expressly defined herein.

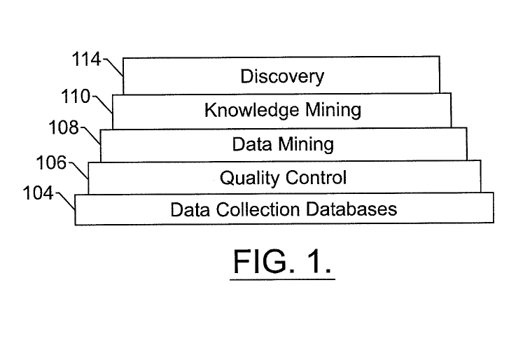

Referring now to FIG. 1, a conceptual overview of environments in which embodiments of the present invention may be used, is shown. As shown in FIG. 1, these environments may include large amounts of data that may be collected in many disparate or independent databases including public, private and/or other databases 104. Each database may have associated therewith a quality control tool 106 that can check for errors, database integrity and/or other parameters within the individual database.

Still referring to FIG. 1, data mining tools may be used as were described above, to allow searching within and/or across databases 104. However, data mining/data warehousing may have shortcomings in integrating and/or querying diverse databases. Moreover, in other embodiments, data mining tools need not be used.

Still referring to FIG. 1, some embodiments of the present invention may provide knowledge mining, using ontology networks, wherein a plurality of databases is integrated, so that new knowledge or discovery 114 may be established by querying the integrated data structure. Accordingly, embodiments of the present invention can provide a knowledge mining layer 110 that can allow virtual discovery 114 to be obtained, based on independent databases 104 that are collected from disparate sources.

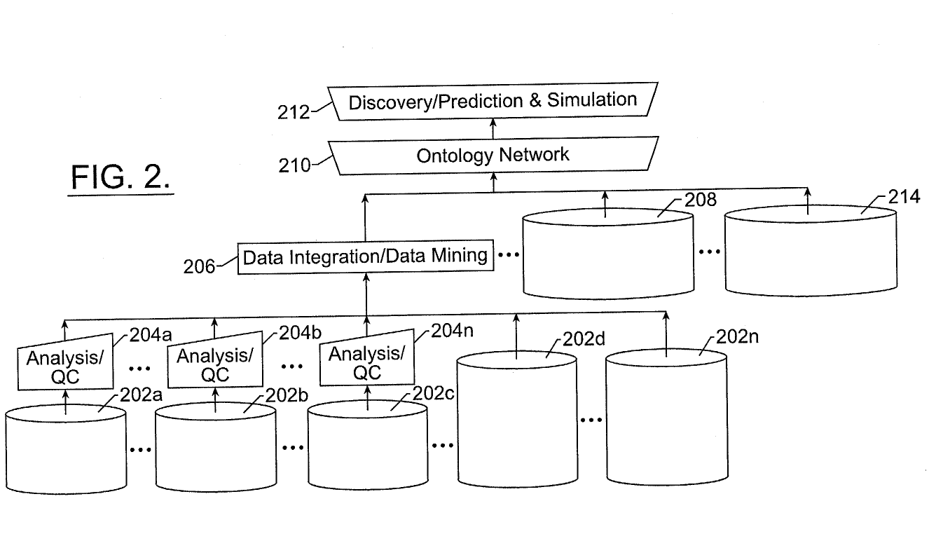

Referring now to FIG. 2, another conceptual overview of environments in which embodiments of the present invention may be used is shown. As shown in FIG. 2, a plurality of disparate databases 202a-202n, 208 and 214 may be provided. More or fewer databases also may be provided, and one or more of these databases may be merged or bifurcated.

Each of these databases 202a-202n, 208 and 214 includes records for a plurality of objects, also referred to herein as entities. These databases 202a-202n, 208 and 214 also generally include an indication of one or more relationships among the various objects, to thereby define an entity-relationship data structure or model for each of the independent databases. The entity-relationship data structure for each database may be thought of as defining an ontology, which provides a vocabulary of terms and some specification of their meaning and/or relationships among one another. These entities and relationships may represent a set of beliefs on the part of the database creator or other individual(s)/organization(s). Thus, the ontology in a given database represents a belief system about the entities and relationships of the data in the database. Some of the databases may constitute a relational database data model that does not explicitly contain entity-relationship data structures. However, entity-relationship data models may be derived from these data models using conventional techniques, in some embodiments of the invention. In other relational database models, one or more entities may be present or derivable, but relationships may not be present or implicit in the data models. According to some embodiments of the invention, these data models can be integrated with other databases that include an ontology, to provide an ontological context for the data model as well.

Referring again to FIG. 2, the databases 202a-202n may be processed in a quality control layer by data analysis/quality control modules 204a, 204b . . . 204n. These data analysis/quality control modules may provide some data curation and determination of clusters of meaningful information. Other databases, such as databases 202d and 202n, may not include an analysis/quality control layer.

Still referring to FIG. 2, in some embodiments, at least some of the raw, compressed and/or qualified data may be incorporated into a warehouse by a data integration/data mining layer 206, which can enable the organization of the data into logically structured tables of information. Data querying may conventionally be performed at the data integration/data mining tool or layer 206, for example by developing specialized query requests to gain inference or knowledge from the warehouse. In other embodiments, a data integration/data mining tool 206 is not used.

In some environments, embodiments of the present invention may operate on top of this data integration/data mining tool 206, and/or may also operate directly on a database, such as the database 208 and/or the database 214. Some embodiments of the present invention can provide a knowledge mining layer in the form of an ontology network 210 that can overlay/merge/associate diverse ontologies that are represented in diverse databases, data tables and/or data repositories. The resulting ontology network 210 thus can link multiple disparate ontologies.

As will be described in more detail below, according to some embodiments of the present invention, an ontology network 210 can incorporate the entity-relationship models of the databases on which it is built, but can also define new relationships or hierarchies by the process of overlay, merge and/or association of entities from the independent ontologies. This conceptualization of knowledge can serve as a specification mechanism for the development of a broad-mesh belief system that can deliver experimental insight. Stated differently, ontology networks 210 according to some embodiments of the present invention can traverse and, thereby, establish a linked path of relationships creating associations between characteristically unlike entities, to thereby allow the revelation of new information and knowledge. The resulting lattice of semantically rich metadata can form an ontology network 210 that captures the knowledge from the data sources 202, 208 it supports.

Thus, as shown in FIG. 2, in some embodiments of the present invention, an ontology network 210 can be located above the data integration layer 206, and can provide a knowledge tool or layer that is available for hypothesis or question-driven mining, as opposed to complex data mining queries that may be typical of data mining applications. Thus, some embodiments of the invention can provide a meta-database of entities and/or relationships that can allow efficient and intelligent analysis of accumulated data.

Still referring to FIG. 2, ontology networks 210 according to some embodiments of the present invention may be linked to an application tool or layer, such as a discovery/prediction and simulation tool 212, so as to allow more accurate discovery, prediction and/or simulation.

Referring now to FIG. 3, a hardware/software block diagram of some embodiments of the present invention now will be described. It will be understood that some embodiments of the present invention may execute on one or more personal, application and/or enterprise computer systems, in a standalone, networked, distributed, pervasive, peer-to-peer and/or other configuration.

Referring now to FIG. 3, a data processing engine 300, which also may be referred to as an ontology engine, can be used to integrate, update and/or query a plurality of databases, and/or generate, add to and/or query an ontology network as will be described in detail below. The engine 300 can provide a knowledge mining layer 110 of FIG. 1 and/or an ontology network 210 of FIG. 2 in some embodiments. The engine 300 is responsive to one or more loaders 302 that can extract relevant information from one or more databases 304, which can be analogous to the data collection layer 104 of FIG. 1 and/or the databases 202, 208 of FIG. 2. In some embodiments, a priori knowledge of the semantics of the ontology that is represented by the associated databases 304 is built into the loader 302 of that ontology's external data files. Moreover, in some embodiments, the loader 302 has knowledge of the semantics of the appropriate part of the engine 300, to which the ontology data connects.

In some embodiments, the engine 300 generates metadata in the form of an overlaid/merged/associated entity-relationship data structure, which can be stored in a metadata database 308. One or more applications 306 may be used for providing discovery, prediction, simulation and/or other applications, analogous to the discovery layer 114 of FIG. 1 or the discovery/prediction and simulation layer 212 of FIG. 2. These applications 306 can interface with a local user interface and/or can interface with a Web browser 316 that is connected to a Web server 312, for example, via a network, such as the Internet 314. The design of a Web server 312, a network such as the Internet 314, and a Web browser 316 is well known to those having skill in the art and need not be described further herein. Finally, user-defined path rules 322 and/or predefined path rules 324 may be provided to allow directed path traversals as will be described in detail below.

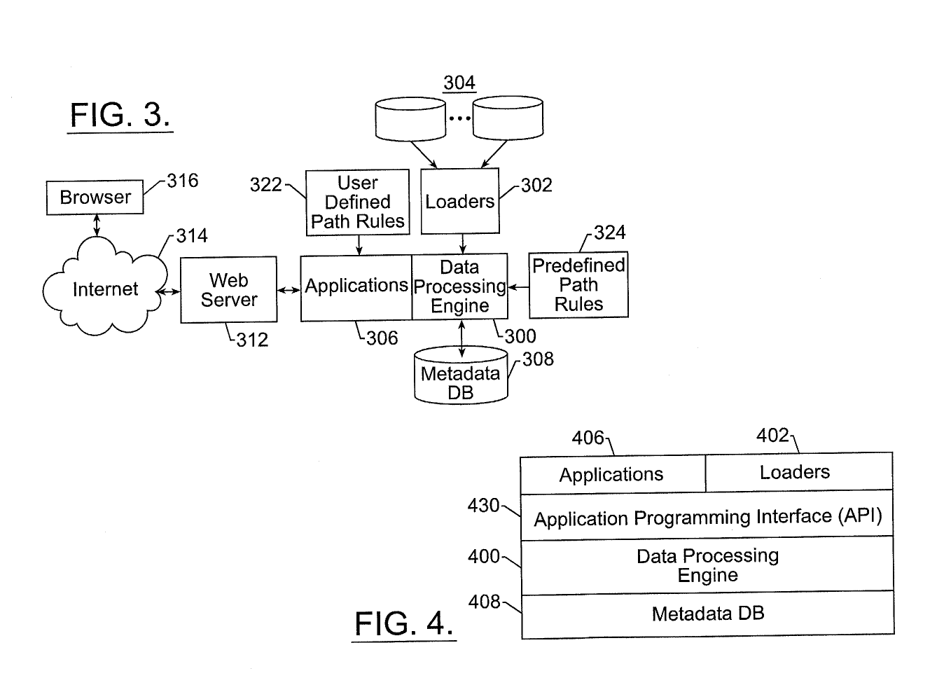

FIG. 4 is a software architecture diagram of some embodiments of the present invention. These embodiments may be used on one or more personal, application and/or enterprise computer systems in a standalone, networked, distributed, pervasive, peer-to-peer and/or other configuration. As shown in FIG. 4, a data processing engine 400 can generate the metadata for a metadata database 408 as will be described in detail below. An Application Programming Interface (API) 430 may be provided to interface the engine 400 with one or more external database loaders 402 and one or more applications 406. The engine 400, metadata database 408, loaders 402 and applications 406 may be analogous to elements 300, 308, 302 and 306, respectively, of FIG. 3.

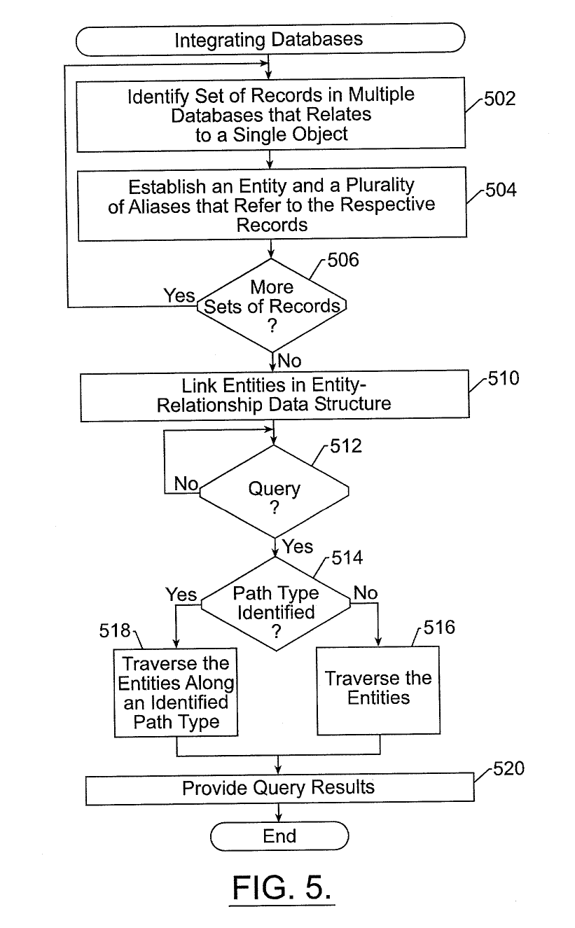

Referring now to FIG. 5, operations for integrating databases according to some embodiments of the present invention now will be described. It will be understood that these operations may be embodied, for example, in a knowledge mining layer 110 of FIG. 1, an ontology network 210 of FIG. 2, an engine 300 of FIG. 3 and/or an engine 400 of FIG. 4. These embodiments can integrate a plurality of disparate or independent databases, such as the databases 202a-202n, 208 and 214 of FIG. 2, and/or 304 of FIG. 3, each of which includes records for a plurality of objects.

Referring now to Block 502, a set of records is identified in the plurality of databases that relates to (i.e., is associated with) a single object. At Block 504, an entity is established in a data structure that corresponds to the single object. The entity includes a plurality of aliases, a respective one of which refers to a respective record in the set of records in the plurality of databases. At Block 506, if there are more records, the operations for identifying and establishing (Blocks 502 and 504, respectively), are repeatedly performed for a plurality of sets of records and, in some embodiments, for all sets of records, in the plurality of databases, to establish a plurality of entities in the data structure.

Still referring to FIG. 5, in other embodiments of the invention, as shown at Block 510, the plurality of entities in the data structure are linked in an entity-relationship model of the plurality of databases. It will be understood that the operations of Block 510 may be performed in parallel with the operations of Block 504, and need not be performed after a plurality or all sets of records have been identified (Block 502) and entities have been established (Block 504).

Still referring to FIG. 5, according to other embodiments of the invention, at Block 512, a query may be received. The query may be received from an application or other program with or without direct user intervention. As shown at Block 514, the query may identify or specify a path type through the entity-relationship model. As shown at Block 516, in some embodiments, if no path type is identified, the plurality of entities that are linked in an entity-relationship model is traversed in response to a query, to thereby obtain query results that are based on the records in the plurality of databases. In contrast, at Block 518, if a path type is identified, the plurality of entities that are linked in an entity-relationship model is traversed along the identified type of path or paths in response to a query, to thereby obtain query results that are based on the records in the plurality of databases. These query results may be provided at Block 520 via an application, such as an application tool 306 of FIG. 3 and/or 406 of FIG. 4. These queries may provide virtual experiments and/or discovery (Blocks 112 and 114 of FIG. 1), and/or discovery/prediction and simulation (Block 212 of FIG. 2). These queries also may represent discovery processes that are recorded and reused.

As will be described in detail below, in some embodiments, the query may specify a starting entity and an ending entity, and the operations of Block 516 can traverse the plurality of entities that are linked in the entity-relationship model from the starting entity to the ending entity, to thereby identify relationships between the starting entity and the ending entity that are based on the entity-relationship model of the plurality of databases. In other embodiments, the entities are traversed from a starting entity to a plurality of ending entities in response to a query that specifies the starting entity, to thereby identify relationships between the starting entity and the plurality of ending entities that are based on the entity-relationship model of the plurality of databases.

Moreover, the path type of Block 514 may be identified using one or more path rules, such as user-defined path rules 322 and/or predefined path rules 324 of FIG. 3. The path rules may specify, for example, a type of path to use in traversing through the plurality of entities, a type of path not to use in traversing through the plurality of entities, a type of ending entity that can be included in the query results, a type of ending entity that is not to be included in the query results, a type of relationship to be used in traversing through the plurality of entities, a type of relationship that is not to be used in traversing through the plurality of entities and/or a confidence level to be achieved in traversing through the plurality of entities. Many other path rules also may be provided.

Finally, when the query results are provided in the Block 520, some embodiments store the query results that are based on the entity-relationship model of the plurality of database, as at least one new relationship is the entity-relationship model. Knowledge that was derived from the query thereby may be stored in the entity-relationship model.

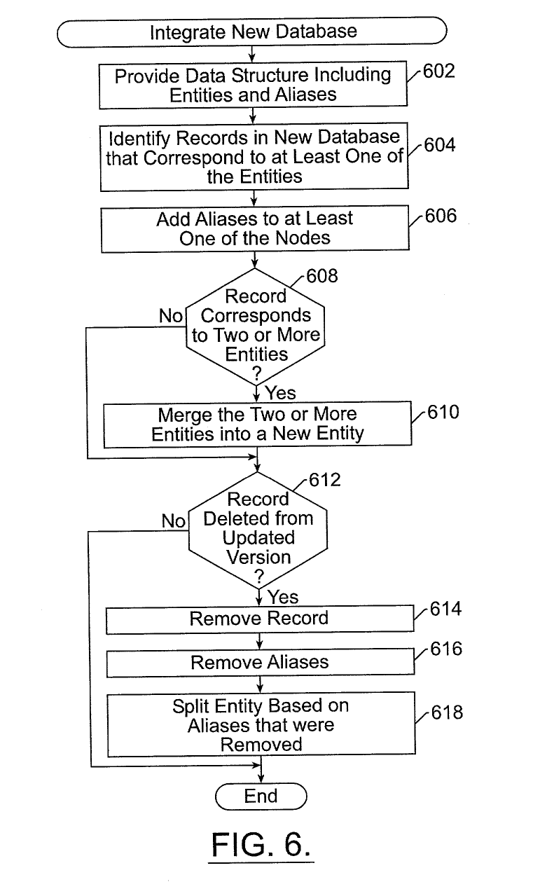

Referring now to FIG. 6, operations for integrating a new database into a plurality of databases, each of which includes records for a plurality of objects, according to some embodiments of the present invention, now will be described. At Block 602, a data structure is provided that includes a plurality of entities, a respective one of which corresponds to a single object. At least some of the entities include a plurality of aliases, a respective one of which refers to a record in a respective one of the plurality of databases that relates to a single object. In some embodiments, the operations of Block 602 may be provided by performing the operations of Blocks 502-510 in FIG. 5. Thus, a preexisting data structure may be provided, and/or a data structure may be generated as was described in FIG. 5.

Referring again to FIG. 6, at Block 604, records are identified in the new database that correspond to at least one of the entities in the existing data structure. In some embodiments, the new database includes an entity-relationship model or an entity-relationship model is generated therefor. In other embodiments, the new database may merely be a relational database data model that does not, explicitly or implicitly, define relationships. By integrating the entity or entities in this new database with the existing entity-relationship model, an ontological context can be provided for the new database. Then, at Block 606, aliases are added to at least one of the entities of the data structure that correspond to the records in the new database, to thereby integrate the new database into the plurality of databases. Thus, additional databases may be readily integrated into the data structure for a plurality of databases.

Referring again to FIG. 6, in other embodiments of the invention, operations may be provided for identifying when a record in the new database corresponds to two or more entities in the existing data structure (Block 608). If this is the case, then at Block 610, the two or more entities in the existing data structure are merged into a new entity that includes aliases that correspond to the records associated with the two or more entities in the data structure, as well as the record in the new database that corresponds to the two or more entities in the data structure. Thus, the data structure can be modified as new databases are incorporated.

Still referring to FIG. 6, operations may be performed according to other embodiments of the present invention, when the new database is an updated version of one of the plurality of databases that already are contained in the data structure. Thus, as shown at Block 612, at least one record in the one of the plurality of databases that has been deleted from the updated version of the one of the plurality of databases is identified. At Block 614, when such a record has been identified, the at least one record is removed from the one of the plurality of databases that has been deleted. At Block 616, aliases that are associated with the at least one record also are removed. Moreover, at Block 618, the at least one entity in the data structure may be split based upon the aliases that were removed. Thus, as new versions of one or more of the databases are incorporated to replace an older version, the data structure may be updated.

In yet other embodiments of the invention, when the data structure is updated by addition, deletion and/or splitting, an image, instance or version of the earlier data structure may be maintained. This image may be used for archival purposes, to ascertain the state of the data structure during a discovery, according to some embodiments of the invention. In other embodiments, comparisons may be made between different images of the data structure, to itself lead to new discovery. Thus, for example, one image of the entity-relationship model can store data related to successful drug discoveries, from genomic to clinical indicators, to extract traversal patterns related to likelihood of success. Another image can store a similar set of patterns for expensive drug failures that did not make it through a genomic, pre-clinical or clinical phase. These images can be compared in order to obtain discovery that can predict success.

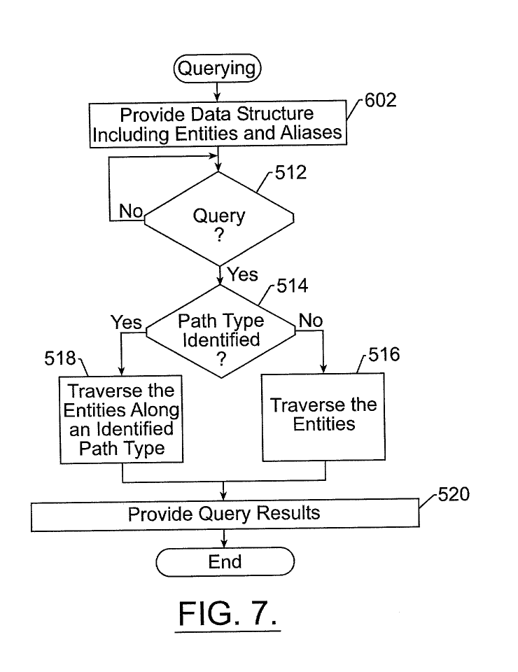

Referring now to FIG. 7, operations for querying a plurality of databases, each of which includes records for a plurality of objects, now will be described according to some embodiments of the present invention. As shown in FIG. 7 at Block 602, a data structure including a plurality of entities and a plurality of aliases, is provided, as already was described in connection with FIG. 6. Then, the plurality of entities that are linked in an entity-relationship model is traversed in response to a query, to thereby obtain query results, for example using operations 512-520 of FIG. 5. These operations will not be described again for the sake of brevity.

Additional qualitative discussion of integration and/or querying of databases according to some embodiments of the present invention that were described in FIGS. 5-7 now will be provided. In particular, some embodiments of the invention can import different types of data from a Tab-Separated-Value (TSV) format, a simple eXtensible Markup Language (XML) format and/or other formats. Scripts may be provided to convert all common data formats to this TSV, XML and/or other formats. Some embodiments can create entities with many different aliases, parents and children. Entities can be merged if they are found to be equivalent. The entities may be organized in Directed Weighted Graph (DWG) based ontologies, as well as hierarchical and/or single level classifications. For non-expert users, a HyperText Markup Language (HTML)-based database viewer, which allows the user to search for terms and then move between different entities via hyperlinks, may be provided. Other embodiments also can produce a tool for traversing across multiple relationships to construct a logical path. Yet other embodiments can provide a tool for importing stored traversals in order to automatically execute those traversals across multiple entities.

Thus, some embodiments of the invention can provide a cross-reference query tool for searching across multiple databases, returning only entities which meet the specified query criteria in all databases. Other embodiments also can provide a translation and annotation tool that can allow translation from one naming system to another naming system, and automatic annotation of data files using different naming systems with description data from differing imported databases. Still other embodiments can provide a clustering engine and viewer, which can allow a user to take clustered experimental data from another program and compare it with data clustered by differing data types (e.g., molecular function) to see how well the experimental clusters predict the annotation clusters and if there are additional annotation clusters. Finally, still other embodiments can provide an unsupervised grouping search, which can take a list of clustered entities and can automatically generate a hypothesis of why they are grouped.

Accordingly, some embodiments of the present invention can bridge the naming system barrier by acquiring information from databases with names of entities residing in multiple repositories, and merging one or many entities as appropriate. Heretofore, lack of merging may have been a barrier to query expansion. In particular, research often includes the understanding that a natural and intuitive relationship exists between entities, and these relationships can be documented to provide a mechanism to build a traversal across multiple such entities, to establish an interpreted or inferred solution. These traversals also can identify a cause and effect relationship. Embodiments of the invention can merge the different names of the identical entities from different unintegrated (independent) data repositories, to thereby allow these traversals to be accomplished. Thus, embodiments of the present invention can apply an integration layer above the disparate data repositories and, therefore, can bind many related data repositories together. These embodiments can enable and promote increased biological context and information mining.

Some embodiments of the invention can generate, expand, update and/or query a data structure containing many nodes, each representing an entity with multiple aliases. Using entity nodes, rather than a different table for each database (as in a star schema), means that all records in diverse databases that represent the same object can be merged into a single entity.

In other embodiments, the entities or nodes are connected by relationships into a DWG, which means that every entity can have multiple children and multiple parents. The DWG allows a single entity to be grouped with other entities by as many different methods as desired, while still allowing these groups to be kept separate from each other.

In other embodiments, the data structure is also designed to be typeless, meaning that, although each entity is associated with a specific category, the same data structure can be used to represent all entities, as well as relationships between them. By using the same data structure, the data structure can potentially store any type of data without any modification. Moreover, some embodiments of the present invention can traverse the DWG unsupervised, so that these embodiments do not need to be told which path to take in order to find relationships or similarities.

Some embodiments of the invention may be implemented in both object oriented and Relational Database Management Systems (RDBMS) models, each of which may have potential advantages. One of the potential advantages of a relational database is that it may be queried with Structured Query Language (SQL). Also, since potential users may already own an RDBMS, deployment can be simpler. If a user does not own an RDBMS there are many systems available. A potential advantage of an object oriented database implementation is that interaction with object-oriented software can be simpler than with an RDBMS.

As was described above, some embodiments of the present invention can identify and merge records in a plurality of databases that represent the same entity. Since identifiers within a naming system are considered to be unique, two objects with the same naming system-identifier pair are considered to be identical. In some embodiments, as was described in connection with Blocks 608 and 610, a record will be added and have an identity cross-reference, also referred to as an alias, to a record that has already been incorporated. When an alias is attached to an entity, some embodiments of the invention can check if the exact naming system-identifier pair is already in use. If it is, the entities are merged together, creating a new entity with all of the relationships, aliases and properties of its component entities.

It also will be understood that databases that are integrated according to some embodiments of the invention can be updated often, in some cases weekly or even daily. If new records are added to the databases, embodiments of the invention can add more entities, aliases and/or relationships. Other embodiments may remove or delete references or entries from databases as was described in Blocks 612-618. Deletion may not be explicit—that is to say, there may be nothing in the data file that states, “Entry ABC was removed”. Instead, the entry may not be present in a subsequent version of the database. Some database vendors may approach this issue by rebuilding the entire database with the new data on a regular basis. Unfortunately, this can break relationship links to private annotations that the user might have added, and may even remove these annotations altogether. The total rebuild also may be time-consuming.

According to some embodiments of the invention, deletion may be handled by tagging every alias and every relationship with the database from which it came (the source) and the date of its last update. When a record is read in, some embodiments of the invention can find the entity to which it points and can check the aliases and relationships to see if any of them have the same source as this record. If any aliases or relationships are found which have the same source, but are not in this record, it is determined that they were removed from the record (Block 612) and they can be removed from the database (Blocks 614 and 616) without the need to impact the data that came from other sources.

Moreover, according to other embodiments of the invention, when deleting a record/alias, a situation may occur where two entities had been merged because of a cross-reference, but this cross-reference is later deleted. In this case, some embodiments of the invention may need to determine whether or not to split the entity into several other entities, and which aliases each should have (Block 618). This determination can be thought of as a graph theory problem, which can be solved by determining the transitive closure of the aliases (as nodes) and the update information (as connections). The existence of a connection between two aliases can be used as an indication that they belong in the same entity. If all the aliases belong in the same entity then a split may not need to be made.

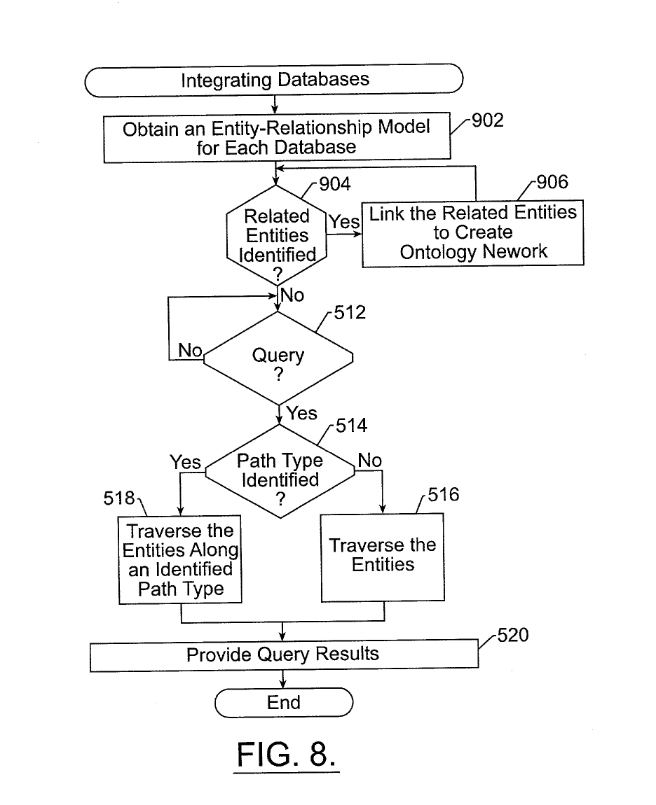

FIG. 8 is a flowchart of operations for integrating databases according to other embodiments of the present invention. As will be described below, these embodiments can create an ontology network from a plurality of independent ontologies, to thereby provide a foundation for discovery.

In particular, referring to FIG. 8 at Block 902, an entity-relationship model is obtained for each of the plurality of databases. It will be understood that the entity-relationship model may be available as part of the database schema of each of the databases so that it merely may need be received. If not, an entity-relationship model may be created using known techniques. Accordingly, the word obtain, as used herein, includes receiving an existing entity-relationship model and/or creating an entity-relationship model.

Then at Block 904, at least some of the related entities in the entity-relationship models in at least two of the databases are identified. At Block 906, the related identities in the entity-relationship models in the at least two of the databases are linked, to thereby create an entity-relationship model that integrates the plurality of databases and creates an ontology network. Operations at Blocks 904 and 906 are repeated until a plurality of related entities, and in some embodiments all related entities, are identified and linked. Once the ontology network is created, a query may be performed by performing operations of Blocks 512-520, as were already described. This description will not be repeated for the sake of brevity.

In some embodiments of the invention, the related entities are identical entities that are linked by merging into a single identity. In other embodiments, the related identities need not be identical. In particular, in some embodiments, entities which are similar but not identical may be associated with one another through a relationship type. The two entities may share aliases, inherit relationships from one another, and may share all benefits of a merge, but may remain separate entities. In other embodiments, entities which are similar but not identical may be associated with one another through a parent entity. All of the identical information may be contained in the parent entity in these embodiments, while the differential information is contained in the child entities. Common relationships are inherited through the parent entity, while relationships particular to the child entities are not. Finally, in still other embodiments, entities which are deemed to be related through traversal may be associated through the construction of a meta-relationship which encapsulates the multiple relationships along the original traversal. Yet other examples of linking of related entities may be provided, according to other embodiments of the invention.

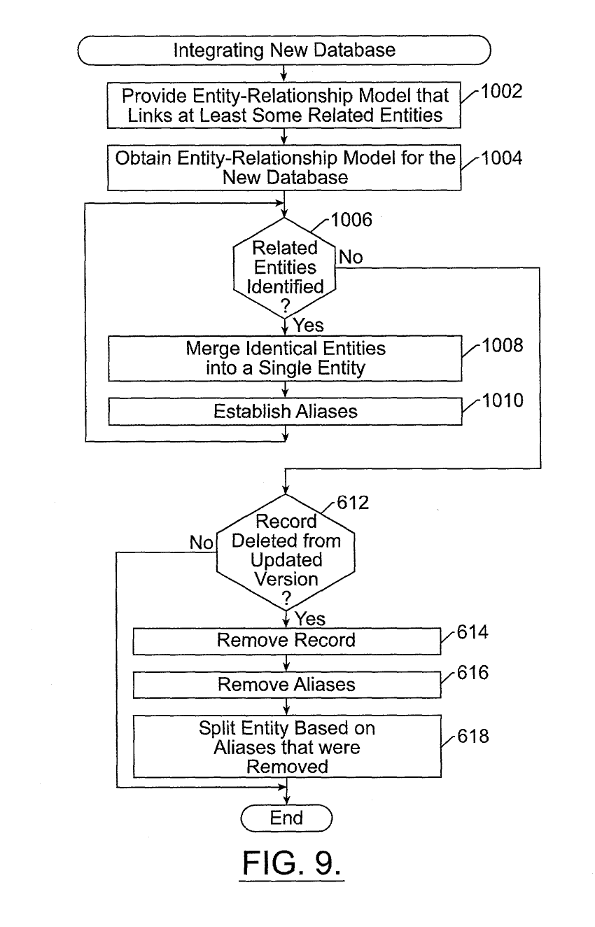

Referring now to FIG. 9, operations for integrating a new database into a plurality of databases according to some embodiments of the invention now will be described. In particular, as shown at Block 1002, an entity-relationship model is provided for the plurality of databases. The entity-relationship model links at least some related entities in at least two of the databases. This entity-relationship model may be obtained, for example, by performing the operations of Blocks 902-906 of FIG. 8.

Still referring to FIG. 9, at Block 1004, an entity-relationship model for the new database is obtained. At Block 1006, at least some of the related entities in the entity-relationship model for the new database and the entity-relationship model for plurality of databases are identified. If related entities are identified at Block 1006, the identical entities in the entity-relationship model for the new database and the entity-relationship model for the plurality of databases are linked.

For example, in some embodiments, at Block 1008, the identical entities in the entity-relationship model for the new database and the entity-relationship model for the plurality of databases are merged into a single entity. Also, in some embodiments, at Block 1010, a plurality of aliases are established for the entity that is merged, a respective one of which points to a respective one of the identical identifies in the entity-relationship models in the at least two of the databases. The identification of related entities, merging and establishing of aliases (Blocks 1006, 1008 and 1010, respectively) are continued, until a plurality, and in some embodiments all, related entities have been identified and linked. Operations for deleting records also may be performed at Block 612-618 as was described above.

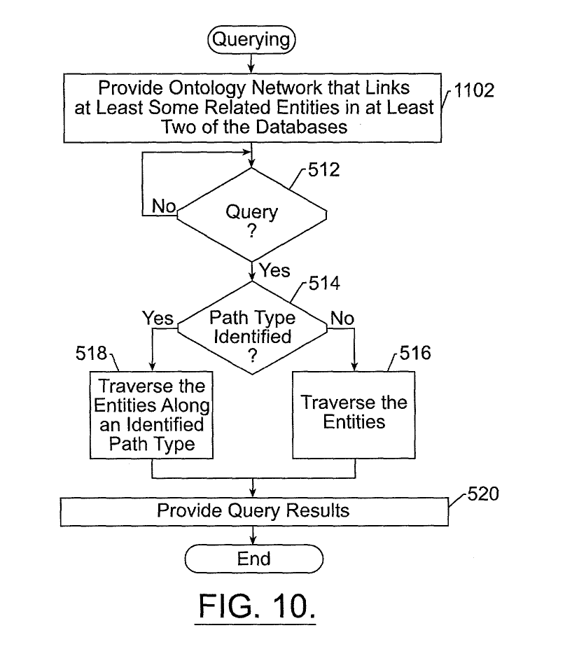

Referring now to FIG. 10, a plurality of databases may be queried according to some embodiments of the present invention, by providing an ontology network that links at least some related entities in at least two of the databases at Block 1102. This ontology network may be provided by performing the operations of FIGS. 8 and/or 9. Querying may be performed by performing the operations of Blocks 512-520. These operations will not be described again for the sake of brevity.

Additional qualitative discussion of creation of an ontology network according to some embodiments of the present invention now will be provided. Some embodiments of the invention can overlay/merge/associate ontologies and provide extensive cross referencing to other existing data bases, data tables, data repositories, and ontologies. According to some embodiments of the invention, the resulting knowledge layer can provide an ontology network where multiple ontologies and various entities have been linked. The ontology network can bridge previously disparate data repositories, bringing structure to a previously amorphous assembly of independent ontologies of entities and relationships.

According to some embodiments of the invention, this ontology network can provide multidirectional characteristics of parent-child relationships. Specifically, the relationships that hold among the objects or entities of an ontology network can be said to have a character where each entity may have another entity from which it was derived or have or is assigned hierarchical characteristics with regard to another entity. However, since an ontology network need not be limited to this form, other new relationships or hierarchies can be created by the process of overlay, merge and/or association of entities from other ontologies of interest. This conceptualization of knowledge may be constructed of knowledge from objects of similar domain and can serve as a specification mechanism for the development of a mesh belief system that can deliver experimental insight. This system may provide for the ability to traverse and thereby establish a linked path of relationships creating associations between characteristically unlike entities and also may provide for the revelation of new information and knowledge. The resulting lattice of semantically rich metadata can form an ontology network that can capture the knowledge from the data sources it supports.

According to some embodiments of the invention, an ontology network 210 can reside as a part of an information stack where enormous quantities of data are collected, for example as was shown in FIG. 2. In some embodiments, the ontology network can be located above a conventional integration tool or layer 206 and can provide a knowledge mining tool or layer 110 that can be available for hypothesis or question-driven mining as opposed to complex data mining queries typical of data mining applications. Some embodiments of the ontology network can comprise a meta database of terms, entities and/or data relationships that can provide for a more efficient and intelligent analysis of accumulated data.

According to other embodiments of the invention, implementation of discovery 212 that employ this ontology network can provide inference engines. As is well known, the components of an expert system are a knowledge base, which may be implemented according to embodiments of the invention by an ontology network 210, and an inference engine which performs reasoning. According to some embodiments, an inference engine or reasoning software application searches and creates rules by determined pattern matching and then establishes new rules and develops forward chaining of rules. Virtual experiments within the subject field of inquiry can be executed which can significantly enhance accuracies and/or have abilities to correlate observations to original predictive behavior with a broader input of related information than previously may be employed.

Inference engines can be made more accurate as a result of the type designation of relationship, building of newly determined relationships, along with the quantification of the confidence and/or validity assigned to these relationships. As will be described below, some embodiments of the invention can assign confidence to different traversals and/or variations in selected paths as they are determined or discovered. This characteristic of an ontology network according to some embodiments of the invention can be further integrated into use by the creator of the virtual experiment to add greater value and relevance to data across the broad span of information among the many domains made available in this semantically rich metadata layer.

As was described above, an ontology can be thought of as a knowledge construct that contains therewithin an answer to a question or a set of beliefs particular to a given domain. The combination of ontologies results in the creation of an ontology network, which can yield answers to questions that were not originally expressed by any of the original ontologies as conceived. Thus, an ontology used to express a belief about system A, and an ontology used to express a belief about system B can be associated together according to embodiments of the present invention, to express belief about systems A and B, but to also answer a new query C. Thus, an ontology network according to some embodiments of the invention can allow a user to form hypotheses about the role of function in process, or of process in function. Many other hypotheses may be formed.

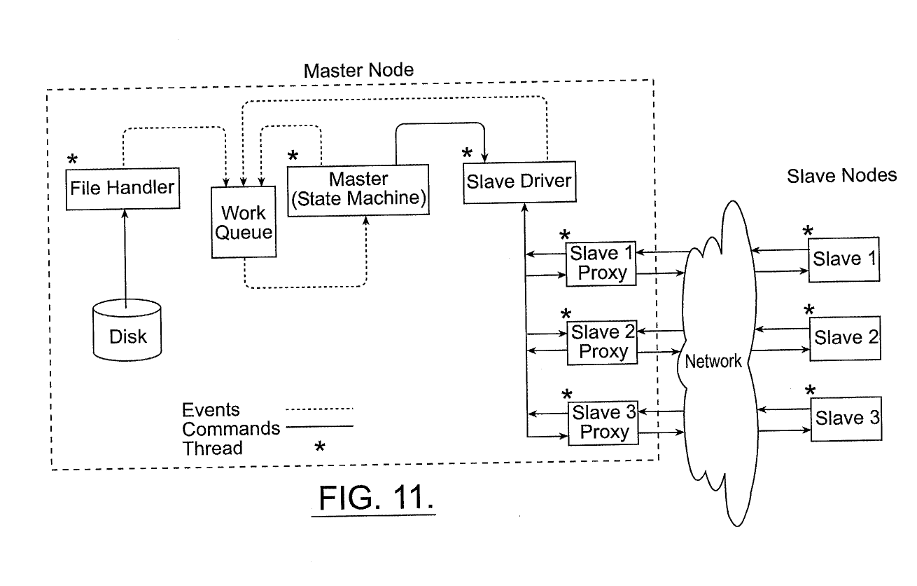

FIG. 11 is a block diagram of a data processing architecture that may be used with some embodiments of the present invention. In particular, the construction of expert systems has been the subject of research in computer science. The creation of a knowledge layer, where a significant responsibility beyond simple reasoning is applied to the inference engine, may need to use supercomputing capabilities. In creating ontology networks according to some embodiments of the present invention, it may be desirable to access significant computing resources. The quantity and time to complete the construction of such an ontology network may be tied to the volume of data in the repositories to be supported by the ontology network and the available computer resources applied during the construction of the metadata referencing the data repositories. Resources ranging from about 30-50 gigaflops may be employed in some embodiments, to construct an ontology network in a reasonable time, such as days. Resources ranging up to about 100 gigaflops or more may be used in some embodiments to construct an ontology network to support larger repositories. A computational system able to support more than 100 Gigaflops of computer power may be among the top 500 supercomputers presently available.

In some embodiments, the creation and/or execution of the ontology network may use peer-to-peer or grid computing technology. Here, processing cycles from many computers on a network are harnessed, and the application used to create the ontology network may be “gridified” to make the best use of these resources. The construction of such a knowledge layer may be well suited to distribution of the millions of small processes. As a result of increasing efficiencies and decreasing costs to employ computer resources as a grid, the construction of such a meta database that captures the information content of the underlying repositories may become a common part of the mining of complex and disparate data systems. The design and operation of peer-to-peer computing systems are well known to those of skill in the art and need not be described further herein.

An example of a database schema which can be used in an ontology network engine, such as an ontology network engine 300 of FIG. 3 or 400 of FIG. 4, to store metadata concerning diverse databases in a metadata database such as the metadata database 308 of FIG. 3 or 408 of FIG. 4, now will be described. It has been found, according to some embodiments of the invention, that the metadata can be stored in a generic database using a conceptual schema that can be implemented using conventional relational database management systems, such as Oracle, MySQL and/or Access.

It will be understood by those having skill in the art that database design may refer to a conceptual schema that exists between the external perception of data (often referred to as an external schema) and the internal on-disk view of data (often referred to as an internal schema). This three-schema architecture conceptualization can enable a programmer to abstract and create various external views of data from the internal view. The conceptual schema can be a composite of all external schemas, such as the use of tables and columns in a spreadsheet, so that external views can be derived from the conceptual schema, while providing the translation for data recording to the physical schema or on-disk structure.

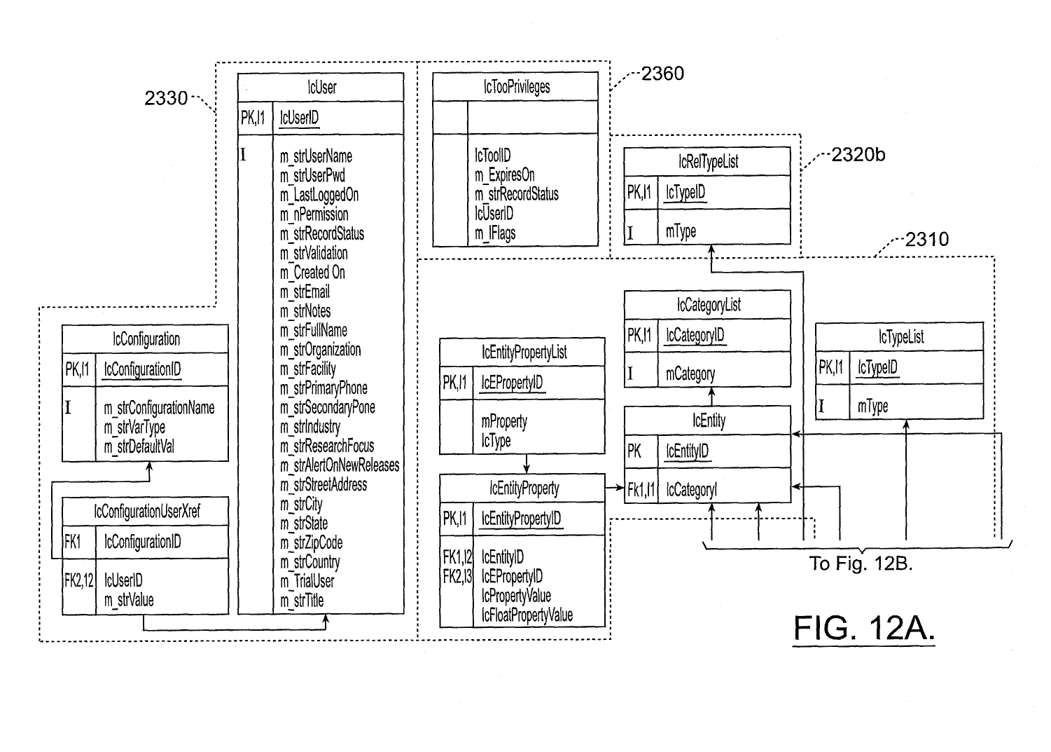

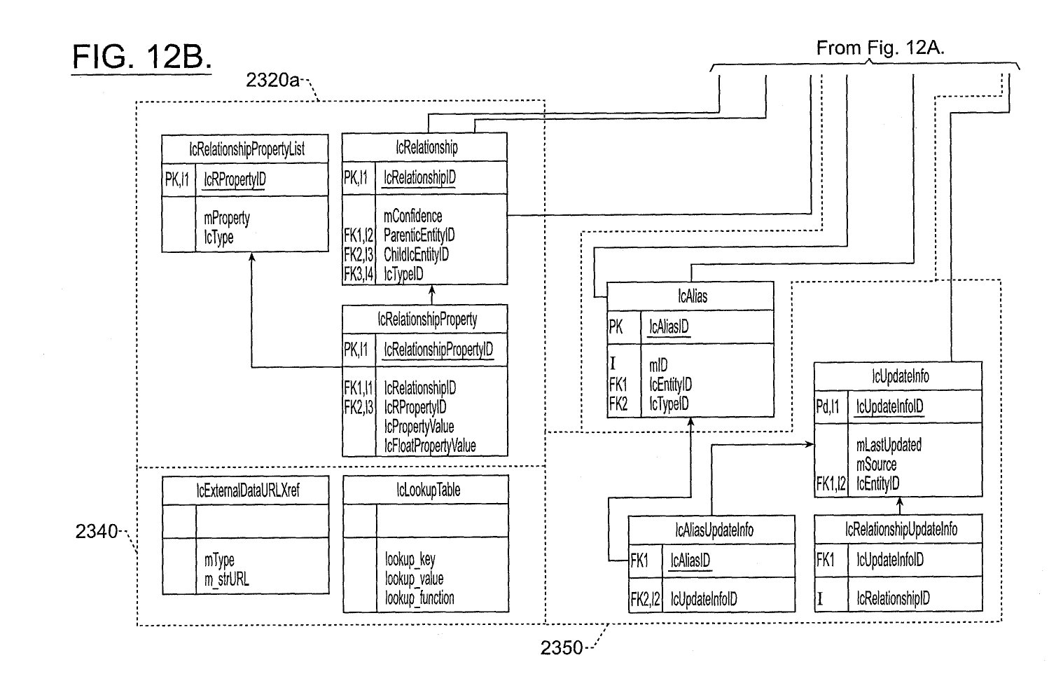

Referring now to FIG. 12, according to some embodiments of the invention, a conceptual schema for an ontology network can itself be embodied as an entity-relationship model. In FIG. 12, the individual boxes may represent tables in a MySQL database. These tables are logical groupings of related data. The lines between the boxes represent relationships between common information or cross-references between distinct tables. The entries inside each box represent unique keys or columns of data for each piece of data held by that table or piece of data.

In particular, referring to FIG. 12, the boxes enclosed by dashed Block 2310 may be used to define entities including the entity name, entity category, attributes or properties of the entity, and aliases of the entities. The boxes enclosed in dashed Blocks 2320a and 2320b may be used to define relationships, including an identification of the relationship, the attributes or properties of the relationship, and the type of the relationship. The boxes enclosed by dashed Block 2330 define user interface aspects including security aspects. The boxes enclosed by dashed Block 2340 define Uniform Resource Locators (URLs) for external databases that may used with an entity browser. The boxes enclosed by dashed Block 2350 provide functionality for updating the ontology when a new version of a database is input. Finally, the box enclosed by dashed Block 2360 defines the applications that can be used with an ontology network. It will be understood that at database schema of FIG. 12 may be used by those having skill in the art to create a relational database using a conventional database management tool.

Thus, the database schema of FIG. 12 is itself represented by an entity-relationship data model. The entities may hold information and may stand alone, or may have relationships between other entities holding data. Thus, the conceptual schema of FIG. 12 illustrates the existing relationships that are declared as being true for the data before discovery of new relationships via inference and/or results are presented. This conceptual schema may be used to create a relational database that can provide a network of ontologies according to some embodiments of the present invention.

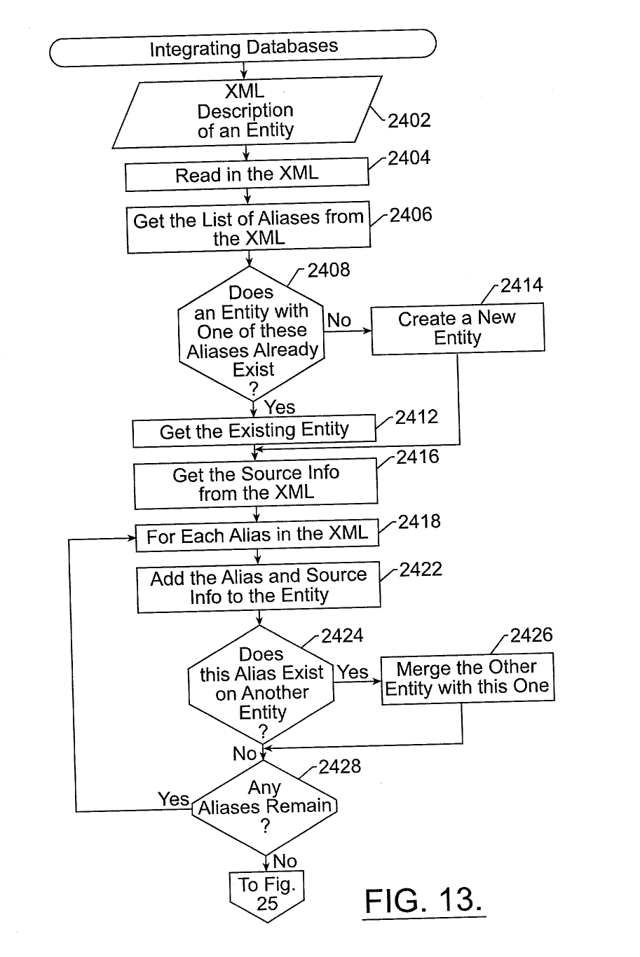

Referring now to FIG. 13, operations for integrating databases and integrating new databases according to other embodiments of the present invention now will be described. These embodiments assume that database records are provided via XML text records. The use of XML text records and the conversion of non-XML records to XML records are well known to those having skill in the art and need not be described further herein. Moreover, it is assumed that the loader, such as the loader 302 of FIG. 3, that is used to load the XML text records also has knowledge of the ontology's semantics based upon the ontology's external data files. As was described above with respect to FIG. 12, the ontology semantics also may be extracted from an external database, if they are not already known. Accordingly, a priori knowledge of the ontology's entities and relationships is known at the time of loading.

Referring now to FIG. 13, operations begin with an XML description of an entity in a database at Block 2402. At Block 2404, the XML description is read. At Block 2406, a list of aliases is obtained from the XML description. At Block 2408, a test is made as to whether an entity with one of these aliases already exists in the network of ontologies. If yes, the existing entity is obtained at Block 2412. If no, at Block 2414, a new entity is created. Source information then is obtained from the XML text at Block 2416.

Continuing with the description of FIG. 13, operations for adding the aliases from the XML input to the entity and merging the entity with other entities when the aliases match now will be described. In particular, for each alias in the XML text file (Block 2418), the alias and the source information are added to the entity at Block 2422. At Block 2424, a test is made as to whether the alias exists in another entity. If yes, the other entity is merged with this one at Block 2426. A test is then made at Block 2428 as to whether any aliases remain and, if so, the operations of Blocks 2418-2426 are repeated until none remain.

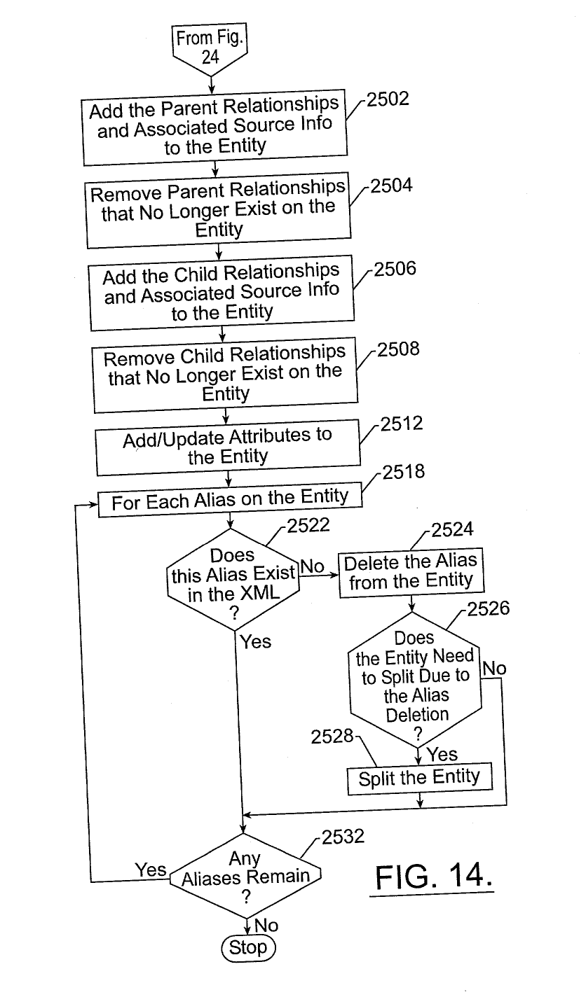

Operations continue at FIG. 14. At Block 2502, parent relationships and associated source information are added to the entity and at Block 2504, parent relationships that no longer exist are removed from the entity. At Block 2506, child relationships and associated source information are added to the entity and at Block 2508, child relationships that no longer exist are removed from the entity. At Block 2512, the attributes are added or updated to the entity.

Still continuing with the description of FIG. 14, operations to remove aliases from the existing entity that no longer appear in the XML input now will be described. In particular, for each alias in the entity (Block 2518), a test is made as to whether this alias exists in the XML text file at Block 2522. If not, the alias is deleted from the entity at Block 2524. Moreover, as a result of deleting the alias from the entity, a test is made at Block 2526 as to whether the entity needs to be split due to the alias deletion and, if so, the entity is split at Block 2528. The operations of Blocks 2518-2528 are completed until there are no aliases left at Block 2532, whereupon operations end.

Accordingly, FIGS. 13 and 14 illustrate operations for inputting data into the ontology network via an XML text record according to some embodiments of the present invention. During these operations, new entities are constructed and merged, to achieve linking and merging of previously disparate entities. The addition of an ontology may be executed in the same manner. In particular, elements of the ontology are read and operations of FIGS. 13 and 14 are followed.

For the purpose of loading an ontology into a preexisting network of ontologies, care may need to be taken because entities within the new ontology may have relationships pointing to other entities within the ontology network, and may also have relationships to entities already existing in the ontology network. The operations that were described above in connection with FIG. 14 can maintain consistency. Thus, FIG. 14 provides embodiments of operations for building new or adding parent and/or child relationships. Removing aliases that may become out of date as a result of an update process also was described. Other new types of relationships, such as reaction right or reaction left or reaction forward or reaction back also may be added, to provide an ability to filter by step.

The following Table describes algorithms that may be used according to some embodiments of the invention, to add an entity and add a relationship using the database schema of FIG. 12 and the operations of FIGS. 13 and 14:

TABLE
Adding an Entity
Overview

Add the entity information.
Add an updateInfo for the entity from the external data source.
Why updateInfos: to differentiate data from different external data
sources in order to handle data inconsistency between those sources.
Once in the system, information cannot be deleted until all external
data sources that put it there agree that it no longer exists.
UpdateInfos are associated with aliases and relationships.
Add Aliases to the entity.
The updateInfo is used when adding aliases.

Add the Entity Information.
Algorithm

Add this entity's category to the category table if it is not
already there.
Add this entity's information to the entity table.
Add this entity's attribute information to the entity property table.

Modified Tables

IcCategoryList
New row added with the entity's category if the category doesn't
already exist.
IcEntity
New row added with the entity's information.
IcEntityProperty
New row(s) added with the entity's attribute information.

Add an UpdateInfo for the Entity from the External Data Source.
Algorithm

If the updateInfo is already in the updateInfo table, update its date
information.
Otherwise, add the updateInfo information to the updateInfo table.

Modified Tables

IcUpdateInfo
New row added with the updateInfo's information.
mLastUpdated column updated with the date information if the
updateInfo is already in the table.

Add Aliases to the Entity
Algorithm

If the alias is already in the database attached to another entity,
then merge that entity with this alias's entity.
This involves taking all the data for the two entities pointed to by the
alias and putting it on a single entity, then removing the other
entity from the system.
Otherwise add the alias's information to the Alias table.
Associate the specified updateInfo with the alias.

Modified Tables
IcAlias

New row added with the alias's information.
IcAliasUpdateInfo
New row added to associate the updateInfo with this alias.
IcTypeList
New row added with the alias's type if the type doesn't already exist.

Modified Tables Due To Merging Entities

IcAlias
IcEntityID column changed to point the alias to the merged entity.
IcEntity
Existing row for the old entity deleted.
IcEntityProperty
Existing row(s) for the old entity attributes deleted.
IcEntityID column updated to point to the merged entity.
IcRelationship
Existing row(s) for relationships on the old entity deleted.
ParentIcEntityID column updated to point to the merged entity.
ChildIcEntityID column updated to point to the merged entity.
IcRelationshipProperty
Existing row(s) for attributes on relationships on the old entity
deleted.
IcRelationshipUpdateInfo
Existing row(s) for updateInfos on relationships on the old entity
deleted.
IcRelationshipID column updated to point to the merged entity.
IcUpdateInfo
IcEntityID column updated to point to the merged entity.

Adding a Relationship
Overview

Add the Relationship.
A relationship is added between two already-existing entities.
One entity is the parent, the other is the child.
Each relationship has an associated UpdateInfo for the external data
source.

Add the Relationship.
Algorithm

If a relationship of this type already exists between the parent and
child, update that relationship's information.
Otherwise add the relationship's information to the relationship table
and its attributes to the relationship attribute table.
Associate the specified updateInfo with the relationship.

Modified Tables

IcRelationship
New row added with the relationship's information.
IcRelationshipProperty
New row(s) added with the relationship's attribute information.
IcRelTypeList
New row added with the alias's type if the type does not already
exist.
IcRelationshipUpdateInfo
New row added to associate the updateInfo with this relationship.

Querying of ontology networks according to other embodiments of the present invention now will be described. In particular, FIGS. 5, 7, 8 and 10 described embodiments for querying the ontology network according to some embodiments of the present invention. However, it will be understood that ontology networks according to some embodiments of the present invention can provide a large number of associations among a large number of entities in diverse ontologies. In some embodiments, discovery may take place by querying the ontology network to traverse the ontology network from one entity to another. Stated differently, in some embodiments, a starting entity and an ending entity may be specified, and the query results can provide some or all of the paths that can link the starting entity to the ending entity, to thereby obtain new discovery.

Unfortunately, due to the large number of linkages between entities that may be provided when building real-world ontology networks, the number of paths which link a starting entity to an ending entity may be inordinately large. In these situations, it may be difficult to obtain discovery by merely traversing the entities, as was described, for example, in Block 516, due to the large volume of related entities and relationships that may be obtained. However, as will now be described, some embodiments of the invention can provide predefined path rules (Block 324 of FIG. 3) and/or user-defined path rules (Block 322 of FIG. 3), and allow traversing the ontology network using these path rules as was described at Blocks 514-520.

More specifically, path rules can specify a type of path to traverse, in response to a given type of query. For example, a path rule may specify a specific type of traversal and a specific type of end point for a specific type of starting point. The path rules can be relatively simple, as was described above, but also can be more complex, involving iterations and/or branching. These path rules can, in effect, create new ontologies within the ontology network based on the belief system of the creator(s) of the predefined or user-defined path rules. A posteriori knowledge of the relationship between the disparate ontologies may be built into the path rules that are developed to traverse the ontology network. Path rules may be devised with specific semantics in mind based on the data loaded into the ontology network. Thus, the relationships generated when a path rule is applied to a specific starting entity can have a well defined meaning.

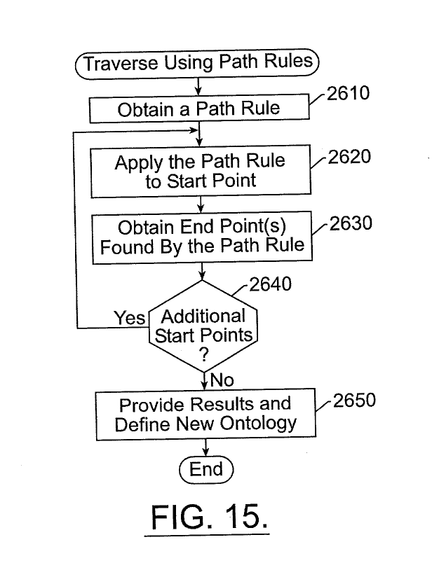

FIG. 15 illustrates operations that may be performed to traverse the entities in an ontology network using path rules, according to some embodiments of the present invention, as was generally described at Block 518. In particular, referring to FIG. 15, at Block 2610, a path rule is obtained either by a user defining a path rule (Block 322), or by obtaining a predefined path rule (Block 324). At Block 2620, the path rule is applied to a specified start point. At Block 2630, the end point or end points found by the path rule are obtained. At Block 2640 a test is made as to whether additional start points are present. If not, at Block 2650, the results of the query may be provided.

Moreover, as also shown in Block 2650, in other embodiments, the start points and end points that are now linked by the path rule can be used to define a new ontology, and can be stored in the metadata database to become a permanent part of the ontology network based upon the belief of the user of the ontology network, rather than merely being a temporary result of a query. In particular, at each step of the traversal through the entities that comprise an ontology network, decisions are made regarding which relationship is selected. Thus, the establishment of a belief at each step or traversal of the system begins to establish multiple steps of order. A decision regarding which step is next in a traversal may be implemented, according to embodiments of the present invention, by providing filtering in the path rules, to thereby create an overall path rule.

Moreover, once a new relationship is declared that is comprised of other steps in the traversal, these rules can be applied by the external schema. Alternatively, they can be physically applied to the internal schema. In other embodiments, a path rule need not persist or be part of the internal schema. Rather, knowledge mining only may need to enable the presentation of this order to the user's results of a study.

At the point of validation of a path, results may yield significant knowledge regarding an entire system of knowledge that is now resident in an ontology network. Thus, with the application of filtering in the path, execution of path rules and/or global filtering according to some embodiments of the present invention, an ontology network can become more than an amorphous set of entities and relationships, and can become more of a rich knowledge base with inherent discoveries therein.

Accordingly, some embodiments of the invention store the query results that are based on the entity-relationship model of the plurality of databases as at least one new relationship in the entity-relationship model, to thereby store knowledge that was derived from the query in the entity-relationship model of the plurality of databases. The ontology network, therefore, can expand based on the knowledge that was obtained as a result of querying the ontology network. In other embodiments, these query results are not stored, so that the query results are not used to modify the ontology network itself.

Filtering according to some embodiments of the invention may specify a relationship type, such as part of, derived from, forward reaction or reverse reaction. Filtering according to other embodiments of the invention also can include or exclude specific types of entities, such as symbols or reactions. Filtering according to yet other embodiments of the invention may also filter on a relationship attribute, entity attribute, alias type, alias ID, category, relationship-type confidence, parent-child, self, and/or other characteristics. Thus, filtering on each step of the traversal can create a preselected path that is acceptable or unacceptable relative to the confidence of the relationship, or as simple as the direction of reaction catalyzed by an agent.

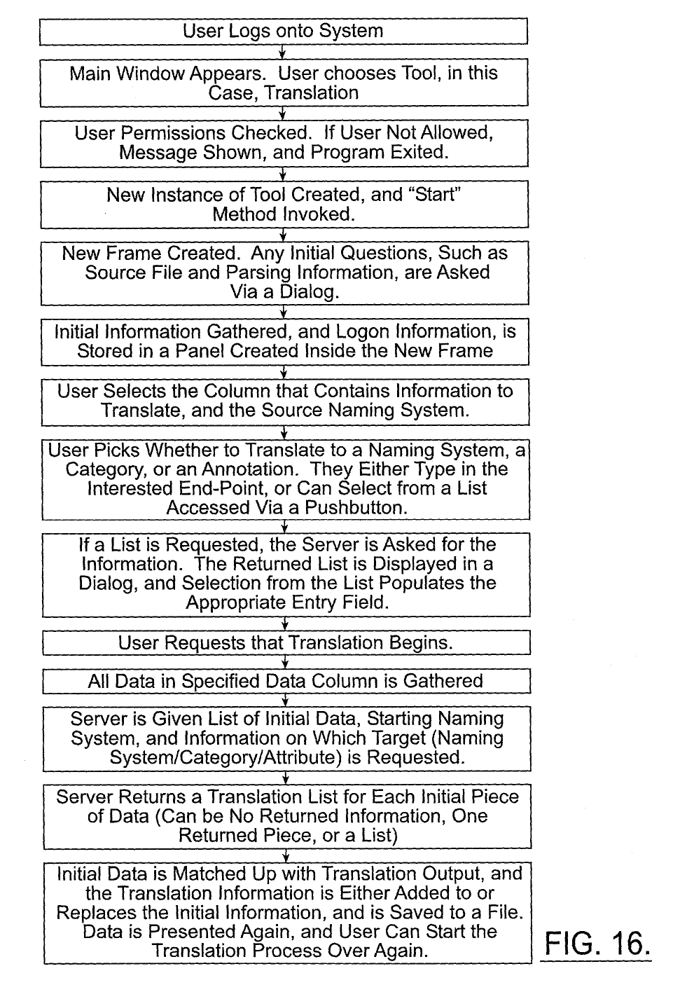

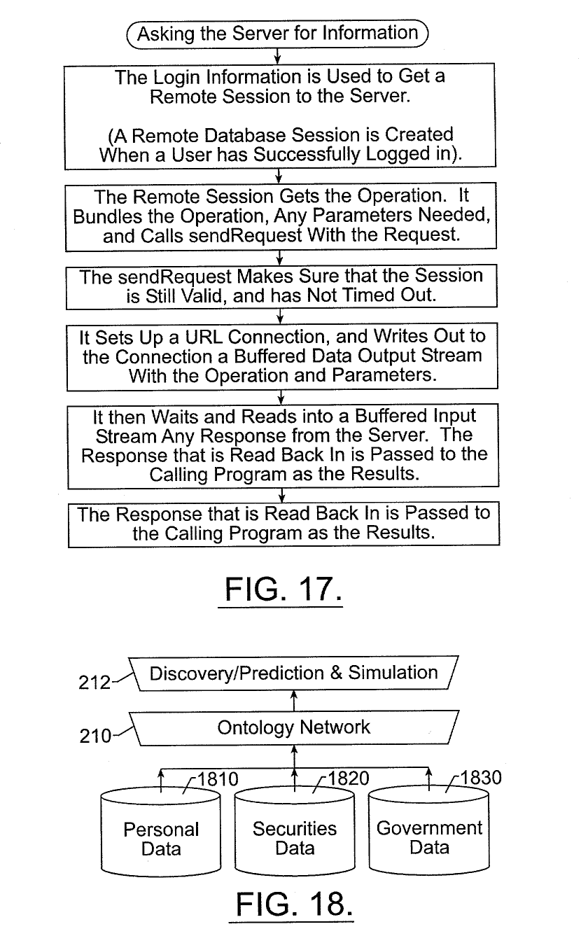

FIGS. 16 and 17 are flowcharts of operations for querying an ontology network according to other embodiments of the present invention. FIG. 16 illustrates querying from a user perspective. FIG. 17 illustrates operations from a client-server standpoint.

According to other embodiments of the present invention, an ontology network can be constructed where the relationships between objects are further labeled and characterized with confidence levels as well as type. The ontology network may be traversed in response to a query, to thereby obtain query results that are based on the entity-relationship model including the at least one confidence level that is assigned. Inferences and correlations commonly employed in the biotechnology area may be characterized to better enable application of these relationships as a more exact and analytical science. This knowledge may not only be harnessed by reasoning engines to create more valid and accurate virtual experiments, but also new relationships may be discovered, built into the ontology network, and/or learned by the ontology network to establish and discover new correlations. The value or quality of these new relationships can be screened and/or further characterized.

In some embodiments of the present invention, information queries of the ontology network can be exact. Results of queries where the retrieved information appears to have been filtered can result from the deployment of knowledge associated with preselected paths. In conventional data queries, data acquired may be filtered to screen unwanted and incorrect results. Not only may this be time consuming, but often the results may still contain significant error and false information. In contrast, queries constructed and run using preselected paths according to some embodiments of the invention may provide only an accurate and concise representation of the information content of the underlying repositories.

In view of the above, some embodiments of the present invention have recognized the principle that relationships between entities may be critical to the discovery process. Embodiments of the present invention can logically organize and cross-reference data into groups, so that the data can be fully accessible and useful. Some embodiments of the invention can merge naming conventions or aliases. Other embodiments of the invention can allow researchers to place proprietary research data into the broadest possible relative context with public research data. Moreover, some embodiments of the present invention can anticipate researchers, think, reduce or eliminate repetitive tasks and/or automate the manual processes that may be used in research and discovery.

Accordingly, some embodiments of the invention can merge redundant database entries from different sources into single entities with alternate names or identifiers. Relationships between entities can capture knowledge from different data sources. These entities and relationships can make up an emergent ontology-based network, capturing the concepts behind databases. This network may not be hard-coded, such that new entity types can be added without the need to modify the underlying database, and relationships between any entities may be allowed. In addition, in many embodiments, entities are sparsely populated, so that only aspects of original data that either involve relationships between entities, or are relevant to user queries may need to be integrated.

Some embodiments of the invention can represent data as entities. Some embodiments of the invention can allow entities to represent any concept or type, including concepts not already represented in the existing entity-relationship model. Because of this, a user can add a completely new concept or type without the need to make changes to the underlying database.

An entity can represent a single concept type or individual of that type. According to some embodiments of the invention, if that concept is present in multiple data sources, the multiple sources are merged into a single entity. In some embodiments of the invention, these database entries can be collapsed into a single entity with the individual identifies as aliases. In practical usage, a user can access all of the relationships for the entity by querying with any of its aliases.

In some embodiments, information about an entity is stored in attributes. In some embodiments, entities can have unlimited attributes, and each attribute has a type and a value. As with entities, attribute types can represent any concept, and new attribute types can be added without the need to make changes to the underlying database. Attributes may store information about an entity for the purposes of searching and filtering, and therefore can be metadata storage containers.

In other embodiments, entities also may be organized into categories or classes, which, like entity types, can be added without the need to change the underlying database. Categories may be used for broad binning of entities.

Some embodiments of the invention may be constructed from databases that have either cross-references to other databases, or lists of alternate names. When a source is imported, entities may be created not only for the source records, but also for the database records they cross-reference. This can be thought of as a virtual database entry. If at a later time that record is loaded, then its information may be added to the entity in some embodiments. In this way, relationships may be built up from multiple sources.

Entity-relationship models according to some embodiments of the invention also can include relationships, which can allow one entity to represent a group of other entities. An entity can be a member of an unlimited number of groups, and each group can represent a different aspect of its members, according to some embodiments of the invention.

Just like entities, relationships can have a type and attributes, in some embodiments of the invention. The type may be used to describe the action of the relationship, while attributes can contain information about the relationship, such as annotation or ontological information (for example, is-a or part-of). Entities can be thought of as nouns, while relationships may be thought of as verbs.

Some relationships may be more certain than others. Therefore, in some embodiments, relationships may have a confidence value to reflect the quality of either the data source or the method used to specify that relationship. Confidence values allow a user to filter out relationships that are of too low quality for their purpose. Because of the confidence values, embodiments of the invention can also be thought of as a DWG.

Some embodiments of the invention can use a specification of rules that define paths using XML. A simple rule is a single step, a path rule is multi-stepped, and a branch rule has conditional branching. A full path may contain different combinations of rule types, and a branch or path rule type can have subrules of any type. In addition, each rule can filter by attribute, type or category. The overall specification of a path defines input and output types or categories.

Some embodiments of the invention also can capture ontological relationships implicitly and/or explicitly. In particular, an entity can explicitly represent an ontological concept. In this case, its parents are more general concepts and its children are more specific concepts. A relationship's type defines how a child concept relates to its parent. Concept entities can also represent groups of instances of that concept.

Some embodiments of the invention also can define an ontology implicitly. In particular, each entity type and category is a concept, while its relationships define the ontological framework. These relationships are built from the cross-references in life science databases. When a new entity type is added, or an entity is put in a relationship with a previously unrelated entity type, new knowledge about how the different entity types relate to each other may be created.

Since an ontology represents a knowledge domain, an entity that has relationships to entities in more than one domain can bridge those domains. In some embodiments, bridge entities are typically experimental or analytical results.

Thus, embodiments of the invention can provide context to independent databases by improving information retrieval, and by enhancing automation and data mining ability. In some embodiments of the invention, new data is merged with existing data, and the resulting entities capture the knowledge and relationships of both sources. Both relationships and entities can have a type for filtering, and attributes for capturing relevant data from original sources. Because of merging and grouping, the resulting ontology network can be more highly connected than the original data sources, which can allow a path to be found between entities in previously unrelated knowledge domains. Moreover, once a path is defined by a user, it can be used in high throughput analyses, such as a microarray results annotation pipeline.

### EXAMPLES

The following examples shall be regarded as merely illustrative and shall not be construed as limiting the invention. The following examples illustrate how three diverse ontologies in the form of databases relating to personal data, securities data and government data can be integrated into an ontology network.

More specifically, referring to FIG. 18, one or more databases related to personal data 1810, one or more databases related to securities data 1820 and one or more databases related to government data 1830 can be integrated into an ontology network 210 by obtaining an entity-relationship model for each of the databases 1810-1830, identifying related entities in the entity-relationship models of at least two of the databases 1810-1830, and linking at least two of the related entities that are identified, to thereby create an entity-relationship model that integrates the plurality of databases. The ontology network 210 may be used for discovery, prediction and simulation 212, as was already described, for example, in connection with FIG. 2.

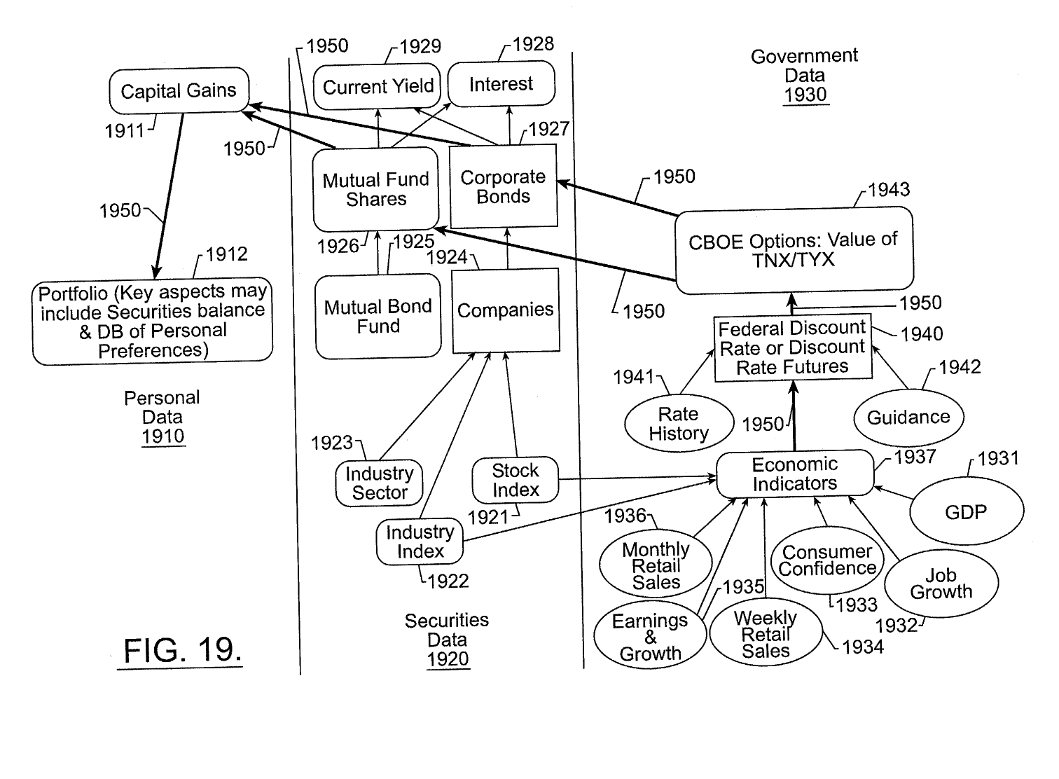

FIG. 19 illustrates a more detailed example of the linking of related entities in entity-relationship models for a plurality of databases. More specifically, FIG. 19 provides a simplified entity-relationship model for a plurality of databases related to personal data 1910, a plurality of databases related to securities data 1920, and a plurality of databases related to government data 1930, which may provide an embodiment of databases 1810-1830, respectively, of FIG. 18.

As illustrated in FIG. 19, the databases related to government data 1930 may include entities for government statistics that may be published on a regular basis, and that constitute databases of economic indicators that can impact options trading of the ten and thirty year government notes which, in turn, can impact the sales of bonds and mutual fund price shares. In particular, entities for Gross Domestic Product (GDP) 1931, job growth 1932, consumer confidence 1933, weekly retail sales 1934, earnings and growth 1935, and monthly retail sales 1936, are related to an economic indicators entity 1937.

As is well known to those having skill in the art, the data in the GDP entity 1931 is a measure of the nation's total output of goods and services. The data in the job growth entity 1932 provides an indicator of whether the job market is expanding or contracting. The data in the consumer confidence entity 1933 is an index of consumer sentiment based on monthly interviews with 5000 households. Weekly retail sales data in entity 1934 is reported by the Census Bureau. The Census Bureau also reports monthly retail sales data in entity 1936. Data for the earnings growth rates entity 1935 is also reported by the federal government.

The entities 1931-1936 are all related to an economic indicators entity 1937. The economic indicators entity 1937 is linked to a federal discount rate or discount rate futures entity 1940 which also includes a rate history entity 1941 and a guidance entity 1942. The federal discount rate or discount rate futures entity 1940 is in turn linked to a conference board options value of TNX/TYX (options on the ten year and thirty year rate) entity 1943. It will be understood that the government data 1930 that is shown at the right-hand side of FIG. 19 represents a simplified entity relationship model of many government databases related to economics.

It also will be understood that government data 1930 generally is tabulated in a number of databases on a large number of related and seemingly unrelated topics. In addition to the entities shown in FIG. 19, other examples include the money in circulation, M1, M2 and M3, and many other such financial numbers. In addition, the government tabulates crop data, weather statistics, weather forecasting, geothermal, geographic, interstellar, gravitational and commodities data. While this data may be relevant to commodity, futures and option trading, such as takes place at the Chicago Mercantile Exchange or the CBOE Exchanges, experts can create relationships or postulate theories of relationships between many of these data types and factors, and their eventual impact on securities markets and/or the value of particular stocks, bonds and mutual funds containing financial instruments of related companies. These expert traversals and/or relationships can be captured in some embodiments of the present invention, for exploitation and application by expert users and/or by less expert users.

Still referring to FIG. 19, an entity relationship model related to securities data 1920 also may be provided. The entity-relationship model related to securities 1920 may include an entity for stock indexes 1921, an entity for industry indexes 1922, and an entity for industry sectors 1923. These entities in turn relate to a companies entity 1924. The companies entity 1924 may be related to a corporate bond entity 1927, which in turn can be related to an interest entity 1928 and a current yield entity 1929. A mutual bond fund entity 1925 may be related to a mutual fund shares entity 1926, which in turn can be related to the interest entity 1928 and the current yield entity 1929.

In particular, many databases exist related to stocks 1921, bonds 1927 and mutual funds 1926. Each of these databases may represent an entity type and may be composed of many different company stocks, bonds or fund shares. An example of an extensive database of this type is the Value Line database of stocks. In this example, Value Line has tabulated about 280 financial characteristics or data items of each company in the list. Their list includes about 6000 different companies in different sectors of the economy. These characteristics can include their proprietary characteristics, such as technical rank and safety rank, and general data such as Beta, relative price-to-earnings ratio, earnings-per-share (current and trailing 12 months), stock price (high/low) and 200 or more other factors that are tabulated for each company. Other related and similar data exist for bonds and mutual funds.

Each of these entity types, as well as each type of stock, bond or mutual fund, may exist in one or more indexes, such as bond indexes, stock indexes and mutual fund indexes. Many of these indexes also are tabulated, and have trading vehicles on the American Stock Exchange, the New York Stock Exchange, or NASDAQ. Many of these entities, such as stocks, bonds, mutual funds and indices, are part of or related to an industry segment. These industry segments have related indexes 1922 and trading vehicles as well.

A particular company sells bonds, sells stocks, creates earnings and is part of mutual funds which also creates earnings, dividends and/or interest. Options (securities derivatives of the above instruments) may be impacted by or tightly related to the underlying securities and react accordingly.

Accordingly, an ontology can be created from the above securities data types 1920, and integration or association of ontologies that can result in the creation of an ontology network according to some embodiments of the present invention. These ontologies may be filled with a great deal of information and relationships, and can be tabulated and stored.

Still referring to FIG. 19, an entity-relationship model related to personal data 1910 may relate to an individual. In particular, a capital gains entity 1911 identifies capital gains in an individual's portfolio, and a portfolio entity 1912 can include a securities balance and a database of personal preferences.

As also shown in FIG. 19, a related entity in at least two of the entity-relationship models is identified. In particular, in FIG. 19, the stock index entity 1921 and industry index entity 1922 in the securities data entity-relationship model 1920 are related to the economic indicators entity 1937 of the government data entity-relationship model 1930. Also, the option entity 1943 is related to the corporate bond entity 1927 and mutual fund shares entity 1926. Finally, the capital gains entity 1911 in the personal data entity-relationship model 1910 is related to the mutual fund share entity 1926 and corporate bond entity 1927 of the securities data entity-relationship model 1920. Thus, at least some of the related entities that are identified are linked, to thereby create an entity-relationship model that integrates the plurality of databases.

A more detailed description of how the integrated entity-relationship model of FIG. 19 may be used by an individual to make portfolio position modifications now will be described. In particular, as also shown in FIG. 19, a path rule may be identified that may link the economic indicators entity 1932 and the portfolio entity 1912 using the relationship path rule 1950 that is shown by bold linking arrows in FIG. 19.

As the economic indicators 1937 change, they can have an effect, by directly impacting the federal discount rate via Federal Reserve Board action, and/or by impacting the perceived federal discount rate futures 1940. This economic data and/or federal action, will impact the CBOE options of TNX and TYX 1943, which are options on the ten year and thirty year Treasury bond rate, and are based on the yield to maturity of the most recently auctioned respective treasury bond. Changes in the value of these instruments are widely watched, and the movement or change in its value can impact the current market, both positively and negatively regarding the sale of corporate bonds 1927. These instruments may change the current yield 1929, and/or may result in further change in value as changes occur in the options market for government securities. Bond fund shares 1925 may also change in value and may further sustain changes in current yield, and/or impact value and cause changes in the interest rates assigned to new issues. Finally, these changes can directly result in a capital gain/loss 1911 from the purchase or sale of these equities. This can impact a portfolio database entity 1912, that includes information on personal preferences of a customer, and an adjustment or rebalancing of a customer portfolio can be recommended.

The above example shows that there can be relationships to a portfolio balance 1912 that can reside not merely in the databases directly associated with the securities data 1920, but that can reach further into information warehouses that are removed from the databases relating to the relevant securities data 1920. This example can be expanded to capture knowledge from those expert in the field that can delineate some or many of the complex relationships that can exist between actions or activities in a global sense that may have a perceived relationship to a portfolio balance, while being remote and/or indirect in a parent/child relationship.

Accordingly, ontology networks, according to some embodiments of the present invention, can be applied to the investment community. In the investment community, investment firms and brokerage houses hire associates to act as portfolio managers or customer client managers. They may have little expert knowledge with regard to the relationships and actions that might indirectly or directly impact particular instruments. Commodity contracts or related security derivatives are examples of such instruments that may be impacted by many peripheral activities or actions that can occur. These actions can include economic, environmental and any other activity, action, event or data that in some way can be related by a combination of traversals to the file, commodity or derivative in question. There presently appears to be a significant need in the securities industry to capture the expert knowledge of the highly experienced investors/traders who may derive their strategies and plans from what could be represented in an ontology network as traversals and association of relationships between key indicators, databases, events, actions and their expected impact on companies and related securities. Embodiments of the invention can allow this expert knowledge to be captured and exploited.

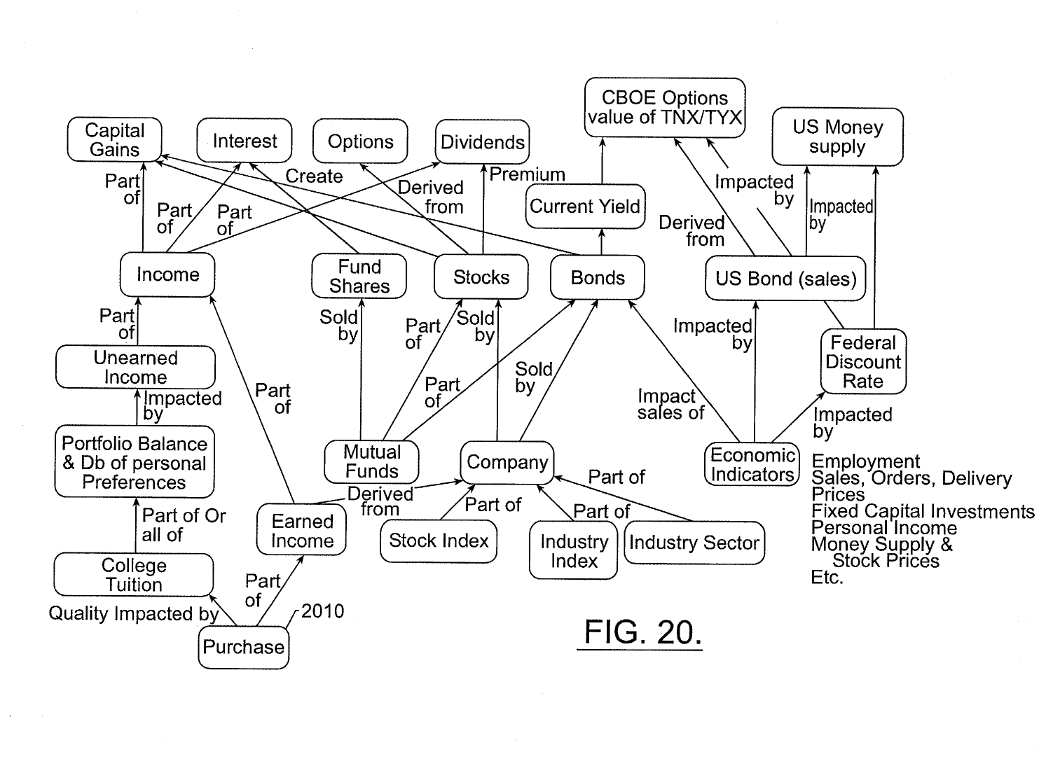

FIG. 20 is a more detailed example of an entity-relationship model that integrates a plurality of databases according to some embodiments of the present invention. In FIG. 20, relationship types also are indicated. This entity-relationship model may be used to obtain expert advice as to the advisability of a major purchase 2010 based on the integration of government data, securities data and personal data. Accordingly, an ontology network that comprises relationships between securities data from companies and information and relationships contained within a number of government databanks, can be used to create a valuable tool to capture expert knowledge in this area for use and application by less skilled industry participants and/or by individuals.

Finally, it will be understood that FIGS. 18-20 provided examples of the integration of personal data, securities data and government data into an ontology network. However, ontology networks may be created in many other fields. Several examples now will be generally described in the fields of criminology/law enforcement, a government budget and the weather. Many other examples may be envisioned by those having skill in the art.

In the field of criminology and law enforcement, data repositories may exist that store retained fingerprint and comparative matching algorithms, DNA data and large databases of information on individuals, where this information on individuals has been generated through either elicit (criminal) activity and/or benign activities, such as public employment. Moreover, local, national and international databases are being developed which include crime scene information and characteristic observations of various crimes. These different ontologies can be merged into an ontology network that could be used, for example, by a task force or other activity whose aim is to understand the nature of organized criminal activity, by integrating the data repositories that are developed on organized crime activities with a host of specific local crime scene information. The relationships that can be established between organized crime activities, national fingerprint databanks, and local crime scene data repositories, can provide an ontology network that can provide new insight into the activities of a criminal organization and/or a clearer focus on their objectives.

In the field of government budgets, it is known that the development of public policy and budgeting for local and national purposes represents a fine balance between the application of funds to various activities relative to public opinion or policies. Accordingly, a relationship may exist between funds that may be available for public welfare, or the creation of new programs, such as a nationally-supported drug subscription plan, and criminal activity on a local, national or international scale. An ontology network, according to some embodiments of the present invention, that integrates international, national and/or local budgetary information and law enforcement data, can be used to provide a predictable understanding of relevant opinion, the results of which may impact other seemingly unrelated programs. This ontology network could be extended to national security, since related data being acquired, as well as the expenses that are entailed, may have an impact on other totally unrelated expenses, and may also have an impact on public opinion and the resulting policy.

As a final example, an ontology network that uses weather data according to some embodiments of the present invention now will be described. In particular, documentation of world weather patterns can enable the prediction of the character and depth of droughts and heavy rain activity. Other global patterns may be observed with regard to development and progress of storms. These data repositories are being accumulated at significant cost worldwide, and include details and analysis of global data, including data relating to the characteristics of a single storm or weather event, as well as generalizations and characteristics of weather events as types. It is further known that weather events can impact crop yields, with the resulting expectations of profits and losses resulting in impacts to certain related futures trading that may also be occurring on global futures markets. Futures trading and changes in the value of futures contracts can impact the resulting decisions by farmers as to their expectations for profit and planting decisions for the next season. While this may directly impact the general food supply, the futures activities may also impact decisions by farm equipment manufacturers to manufacture farm equipment, which is turn can impact raw materials costs and future buying patterns of commercial buyers in industries related to these material acquisitions. An ontology network according to some embodiments of the present invention can merge ontologies related to weather, crops data, futures trading, farm equipment manufacturing and raw materials. This ontology network then can be traversed by an expert, to establish a path rule for retention of the expert knowledge. Thus, expert thinking can be captured to create a representation that can clearly identify the impact of weather on the cost of steel for increased farm equipment production in the coming year, as an example.

In the drawings and specification, there have been disclosed typical preferred embodiments of the invention and, although specific terms are employed, they are used in a generic and descriptive sense only and not for purposes of limitation, the scope of the invention being set forth in the following claims.

What is claimed is:

1. A method of integrating a plurality of databases, comprising:
obtaining an entity-relationship model for each of the plurality of databases;
identifying related entities in the entity-relationship models of at least two of the databases; and
linking at least two of the related entities that are identified, to thereby create an entity-relationship model that integrates the plurality of databases.

2. A method according to claim 1 wherein at least one of the plurality of databases represents an ontology and wherein the entity-relationship model that integrates the plurality of databases creates an ontology network.

3. A method according to claim 1 wherein the related entities are identical entities and wherein linking comprises merging the at least two of the identical entities that are identified into a single entity in the entity-relationship model that integrates the plurality of databases.

4. A method according to claim 3 wherein the merging further comprises establishing a plurality of aliases for the single entity in the entity-relationship model that integrates the plurality of databases, a respective alias of which refers to a respective one of the at least two of the identical entities that are identified.

5. A method according to claim 1 further comprising:
traversing the entity-relationship model that integrates the plurality of databases in response to a query to thereby obtain query results that are based on the entity-relationship model that integrates the plurality of databases.

6. A method according to claim 5 wherein the traversing comprises:
traversing the entity-relationship model that integrates the plurality of databases from a starting entity to an ending entity in response to a query that specifies the starting entity and the ending entity to thereby identify relationships between the starting entity and the ending entity that are based on the entity-relationship model that integrates the plurality of databases.

7. A method according to claim 5 wherein the traversing comprises:
traversing the entity-relationship model that integrates the plurality of databases from a starting entity to a plurality of ending entities in response to a query that specifies the starting entity to thereby identify relationships between the starting entity and the plurality of ending entities that are based on the entity-relationship model that integrates the plurality of databases.

8. A method according to claim 5 wherein the traversing comprises:
traversing the entity-relationship model that integrates the plurality of databases in response to a query and in response to at least one path rule to thereby obtain query results that are based on the entity-relationship model that integrates the plurality of databases.

9. A method according to claim 8 wherein the at least one path rule specifies a type of path to use in traversing through the entity-relationship model that integrates the plurality of databases, a type of path not to use in traversing through the entity-relationship model that integrates the plurality of databases, a type of ending entity that can be included in the query results, a type of ending entity that is not to be included in the query results, a type or class of relationship to be used in traversing through the entity-relationship model that integrates the plurality of databases, a type or class of relationship that is not to be used in traversing through the entity-relationship model that integrates the plurality of databases and/or a confidence level to be achieved in traversing through the entity-relationship model that integrates the plurality of databases.

10. A method according to claim 8 further comprising storing the query and the path rule for reuse.

11. A method according to claim 5 further comprising:
storing the query results that are based on the entity-relationship model that integrates the plurality of databases as at least one new relationship in the entity-relationship model that integrates the plurality of databases to thereby store knowledge that was derived from the query in the entity-relationship model that integrates the plurality of databases.

12. A method according to claim 5 further comprising:
assigning a confidence level to at least one of the relationships in the entity-relationship model that integrates the plurality of databases.

13. A method according to claim 12 further comprising:
traversing the entity-relationship model that integrates the plurality of databases in response to a query to thereby obtain query results that are based on the entity-relationship model that integrates the plurality of databases including the at least one confidence level that is assigned.

14. A method of integrating a new database with a plurality of databases, comprising:
providing an entity-relationship model of the plurality of databases that links at least some related entities in at least two of the databases;
obtaining an entity-relationship model of the new database;
identifying related entities in the entity-relationship model of the new database and the entity-relationship model of the plurality of databases; and
linking at least two of the related entities that are identified, to thereby create an entity-relationship model that integrates the plurality of databases and the new database.

15. A method according to claim 14 wherein the entity-relationship model of the plurality of databases that links at least some related entities in the at least two of the databases provides an ontology network and wherein the entity-relationship model for the new database represents an ontology.

16. A method according to claim 14 wherein the related entities are identical entities and wherein the linking comprises merging the at least two of the identical entities that are identified into a single entity in the entity-relationship model that integrates the plurality of databases and the new database.

17. A method according to claim 16 wherein the merging further comprises establishing a plurality of aliases for the single entity in the entity-relationship model that integrates the plurality of databases and the new database, a respective alias of which refers to a respective one of the at last two of the identical entities that are identified.

18. A method according to claim 17 wherein the new database is an updated version of one of the plurality of databases, the method further comprising:
identifying at least one entity in the one of the plurality of databases that has been deleted from the updated version of the one of the plurality of databases; and
removing an alias that is associated with the at least one entity that has been removed.

19. A method according to claim 18 further comprising:
splitting at least one entity in the entity-relationship model that integrates the plurality of databases and the new database based upon the alias that was removed.

20. A method according to claim 14 further comprising:
identifying entities in the new database that do not correspond to at least one of the entities in the entity-relationship model that integrates the plurality of databases and the new database; and
adding at least one new entity to the entity-relationship model that integrates the plurality of databases and the new database that corresponds to the entities in the new database that do not correspond to at least one of the entities in the entity-relationship model that integrates the plurality of databases and the new database.

21. A method according to claim 14 further comprising:
traversing the entity-relationship model that integrates the plurality of databases and the new database in response to a query to thereby obtain query results that are based on the entity-relationship model that integrates the plurality of databases and the new database.

22. A method according to claim 14 further comprising:
traversing the entity-relationship model that integrates the plurality of databases and the new database in response to a query and in response to at least one path rule to thereby obtain query results that are based on the entity-relationship model that integrates the plurality of databases and the new database.

23. A method according to claim 21 further comprising:
storing the query results that are based on the entity-relationship model that integrates the plurality of databases and the new database as at least one new relationship in the entity-relationship model that integrates the plurality of databases and the new database to thereby store knowledge that was derived from the query in the entity-relationship model that integrates the plurality of databases and the new database.

24. A method according to claim 14 further comprising:
maintaining an image of the entity-relationship model of the plurality of databases prior to the linking.

25. A method according to claim 24 further comprising:
comparing the image of the entity-relationship model of the plurality of databases prior to the linking and the entity-relationship model that integrates the plurality of databases and the new database.

26. A method according to claim 14 wherein the entity-relationship model of the new database does not include relationships therein.

27. A method of querying a plurality of databases, each of which includes records for a plurality of entities, the method comprising:
providing an integrated entity-relationship model of the plurality of databases that links at least some related entities in at least two of the databases; and
traversing the integrated entity-relationship model of the plurality of databases in response to a query to thereby obtain query results that are based on the integrated entity-relationship model of the plurality of databases.

28. A method according to claim 27 wherein the traversing comprises:
traversing the integrated entity-relationship model of the plurality of databases from a starting entity to an ending entity in response to a query that specifies the starting entity and the ending entity to thereby identify relationships between the starting entity and the ending entity that are based on the integrated entity-relationship model of the plurality of databases.

29. A method according to claim 27 wherein the traversing comprises:
traversing the integrated entity-relationship model of the plurality of databases from a starting entity to a plurality of ending entities in response to a query that specifies the starting entity to thereby identify relationships between the starting entity and the plurality of ending entities that are based on the integrated entity-relationship model of the plurality of databases.

30. A method according to claim 27 wherein the traversing comprises:
traversing the integrated entity-relationship model of the plurality of databases in response to a query and in response to at least one path rule to thereby obtain query results that are based on the integrated entity-relationship model of the plurality of databases.

31. A method according to claim 30 wherein the at least one path rule specifies a type of path to use in traversing through the plurality of entities, a type of path not to use in traversing through the plurality of entities, a type of ending entity that can be included in the query results, a type or class of ending entity that is not to be included in the query results, a type or class of relationship that is to be used in traversing through the plurality of entities, a type of relationship not to be used in traversing through the plurality of entities and/or a confidence level to be achieved in traversing through the plurality of entities.

32. A method according to claim 30 further comprising storing the query and the path rule for reuse.

33. A method according to claim 27 further comprising:
storing the query results that are based on the integrated entity-relationship model of the plurality of databases as at least one new relationship in the integrated entity-relationship model of the plurality of databases to thereby store knowledge that was derived from the query in the integrated entity-relationship model of the plurality of databases.

34. A method according to claim 27 further comprising:
assigning a confidence level to at least one of the relationships in the integrated entity-relationship model of the plurality of databases.

35. A method according to claim 34 further comprising:
traversing the integrated entity-relationship model of the plurality of databases in response to a query to thereby obtain query results that are based on the integrated entity-relationship model of the plurality of databases including the at least one confidence level that is assigned.

36. A system for integrating a plurality of databases, comprising:
an entity-relationship model for each of the plurality of databases;
means for identifying related entities in the entity-relationship models of at least two of the databases; and
means for linking at least two of the related entities that are identified, to thereby create an entity-relationship model that integrates the plurality of databases.

37. A system according to claim 36 wherein at least one of the plurality of databases represents an ontology and wherein the entity-relationship model that integrates the plurality of databases creates an ontology network.

38. A system according to claim 36 wherein the related entities are identical entities and wherein the means for linking comprises means for merging the at least two of the identical entities that are identified into a single entity in the entity-relationship model that integrates the plurality of databases.

39. A system according to claim 38 wherein the means for merging further comprises means for establishing a plurality of aliases for the single entity in the entity-relationship model that integrates the plurality of databases, a respective alias of which refers to a respective one of the at least two of the identical entities that are identified.

40. A system according to claim 36 further comprising:
means for traversing the entity-relationship model that integrates the plurality of databases in response to a query to thereby obtain query results that are based on the entity-relationship model that integrates the plurality of databases.

41. A system according to claim 40 wherein the means for traversing comprises:
means for traversing the entity-relationship model that integrates the plurality of databases from a starting entity to an ending entity in response to a query that specifies the starting entity and the ending entity to thereby identify relationships between the starting entity and the ending entity that are based on the entity-relationship model that integrates the plurality of databases.

42. A system according to claim 40 wherein the means for traversing comprises:
means for traversing the entity-relationship model that integrates the plurality of databases from a starting entity to a plurality of ending entities in response to a query that specifies the starting entity to thereby identify relationships between the starting entity and the plurality of ending entities that are based on the entity-relationship model that integrates the plurality of databases.

43. A system according to claim 40 wherein the means for traversing comprises:
means for traversing the entity-relationship model that integrates the plurality of databases in response to a query and in response to at least one path rule to thereby obtain query results that are based on the entity-relationship model that integrates the plurality of databases.

44. A system according to claim 43 wherein the at least one path rule specifies a type of path to use in traversing through the entity-relationship model that integrates the plurality of databases, a type of path not to use in traversing through the entity-relationship model that integrates the plurality of databases, a type of ending entity that can be included in the query results, a type of ending entity that is not to be included in,the query results, a type or class of relationship to be used in traversing through the entity-relationship model that integrates the plurality of databases, a type or class of relationship that is not to be used in traversing through the entity-relationship model that integrates the plurality of databases and/or a confidence level to be achieved in traversing through the entity-relationship model that integrates the plurality of databases.

45. A system according to claim 43 further comprising means for storing the query and the path rule for reuse.

46. A system according to claim 40 further comprising:
means for storing the query results that are based on the entity-relationship model that integrates the plurality of databases as at least one new relationship in the entity-relationship model that integrates the plurality of databases to thereby store knowledge that was derived from the query in the entity-relationship model that integrates the plurality of databases.

47. A system according to claim 40 further comprising:
means for assigning a confidence level to at least one of the relationships in the entity-relationship model that integrates the plurality of databases.

48. A system according to claim 47 further comprising:
means for traversing the entity-relationship model that integrates the plurality of databases in response to a query to thereby obtain query results that are based on the entity-relationship model that integrates the plurality of databases including the at least one confidence level that is assigned.

49. A system for integrating a new database with a plurality of databases, comprising:
an entity-relationship model of the plurality of databases that links at least some related entities in at least two of the databases;
an entity-relationship model of the new database;
means for identifying related entities in the entity-relationship model of the new database and the entity-relationship model of the plurality of databases; and
means for linking at least two of the related entities that are identified, to thereby create an entity-relationship model that integrates the plurality of databases and the new database.

50. A system according to claim 49 wherein the entity-relationship model of the plurality of databases that links at least some related entities in the at least two of the databases provides an ontology network and wherein the entity-relationship model for the new database represents an ontology.

51. A system according to claim 49 wherein the related entities are identical entities and wherein the means for linking comprises means for merging the at least two of the identical entities that are identified into a single entity in the entity-relationship model that integrates the plurality of databases and the new database.

52. A system according to claim 51 wherein the means for merging further comprises means for establishing a plurality of aliases for the single entity in the entity-relationship model that integrates the plurality of databases and the new database, a respective alias of which refers to a respective one of the at last two of the identical entities that are identified.

53. A system according to claim 52 wherein the new database is an updated version of one of the plurality of databases, the system further comprising:
means for identifying at least one entity in the one of the plurality of databases that has been deleted from the updated version of the one of the plurality of databases; and
means for removing an alias that is associated with the at least one entity that has been removed.

54. A system according to claim 53 further comprising:
means for splitting at least one entity in the entity-relationship model that integrates the plurality of databases and the new database based upon the alias that was removed.

55. A system according to claim 49 further comprising:
means for identifying entities in the new database that do not correspond to at least one of the entities in the entity-relationship model that integrates the plurality of databases and the new database; and
means for adding at least one new entity to the entity-relationship model that integrates the plurality of databases and the new database that corresponds to the entities in the new database that do not correspond to at least one of the entities in the entity-relationship model that integrates the plurality of databases and the new database.

56. A system according to claim 49 further comprising:
means for traversing the entity-relationship model that integrates the plurality of databases and the new database in response to a query to thereby obtain query results that are based on the entity-relationship model that integrates the plurality of databases and the new database.

57. A system according to claim 49 further comprising:
means for traversing the entity-relationship model that integrates the plurality of databases and the new database in response to a query and in response to at least one path rule to thereby obtain query results that are based on the entity-relationship model that integrates the plurality of databases and the new database.

58. A system according to claim 56 further comprising:
means for storing the query results that are based on the entity-relationship model that integrates the plurality of databases and the new database as at least one new relationship in the entity-relationship model that integrates the plurality of databases and the new database to thereby store knowledge that was derived from the query in the entity-relationship model that integrates the plurality of databases and the new database.

59. A system according to claim 49 further comprising:
means for maintaining an image of the entity-relationship model of the plurality of databases before the at least two of the related entities are linked.

60. A system according to claim 54 further comprising:
means for comparing the image of the entity-relationship model of the plurality of databases before the at least two of the related entities are linked and the entity-relationship model that integrates the plurality of databases and the new database.

61. A system according to claim 49 wherein the entity-relationship model of the new database does not include relationships therein.

62. A system for querying a plurality of databases, each of which includes records for a plurality of entities, the system comprising:
an integrated entity-relationship model of the plurality of databases that links at least some related entities in at least two of the databases; and
means for traversing the integrated entity-relationship model of the plurality of databases in response to a query to thereby obtain query results that are based on the integrated entity-relationship model of the plurality of databases.

63. A system according to claim 62 wherein the means for traversing comprises:
means for traversing the integrated entity-relationship model of the plurality of databases from a starting entity to an ending entity in response to a query that specifies the starting entity and the ending entity to thereby identify relationships between the starting entity and the ending entity that are based on the integrated entity-relationship model of the plurality of databases.

64. A system according to claim 62 wherein the means for traversing comprises:
means for traversing the integrated entity-relationship model of the plurality of databases from a starting entity to a plurality of ending entities in response to a query that specifies the starting entity to thereby identify relationships between the starting entity and the plurality of ending entities that are based on the integrated entity-relationship model of the plurality of databases.

65. A system according to claim 62 wherein the means for traversing comprises:
means for traversing the integrated entity-relationship model of the plurality of databases in response to a query and in response to at least one path rule to thereby obtain query results that are based on the integrated entity-relationship model of the plurality of databases.

66. A system according to claim 65 wherein the at least one path rule specifies a type of path to use in traversing through the plurality of entities, a type of path not to use in traversing through the plurality of entities, a type of ending entity that can be included in the query results, a type of ending entity that is not to be included in the query results, a type or class of relationship that is to be used in traversing through the plurality of entities, a type or class of relationship not to be used in traversing through the plurality of entities and/or a confidence level to be achieved in traversing through the plurality of entities.

67. A system according to claim 65 further comprising storing the query and the path rule for reuse.

68. A system according to claim 62 further comprising:
means for storing the query results that are based on the integrated entity-relationship model of the plurality of databases as at least one new relationship in the integrated entity-relationship model of the plurality of databases to thereby store knowledge that was derived from the query in the integrated entity-relationship model of the plurality of databases.

69. A system according to claim 62 further comprising:
means for assigning a confidence level to at least one of the relationships in the integrated entity-relationship model of the plurality of databases.

70. A system according to claim 69 further comprising:
means for traversing the integrated entity-relationship model of the plurality of databases in response to a query to thereby obtain query results that are based on the integrated entity-relationship model of the plurality of databases including the at least one confidence level that is assigned.

71. A computer program product that is configured to integrate a plurality of databases, the computer program product comprising a computer usable storage medium having computer-readable program code embodied in the medium, the computer-readable program code comprising:
computer-readable program code that is configured to obtain an entity-relationship model for each of the plurality of databases;
computer-readable program code that is configured to identify related entities in the entity-relationship models of at least two of the databases; and
computer-readable program code that is configured to link at least two of the related entities that are identified, to thereby create an entity-relationship model that integrates the plurality of databases.

72. A computer program product according to claim 71 wherein at least one of the plurality of databases represents an ontology and wherein the entity-relationship model that integrates the plurality of databases creates an ontology network.

73. A computer program product according to claim 71 wherein the related entities are identical entities and wherein the computer-readable program code that is configured to link comprises computer-readable program code that is configured to merge the at least two of the identical entities that are identified into a single entity in the entity-relationship model that integrates the plurality of databases.

74. A computer program product according to claim 73 wherein the computer-readable program code that is configured to merge further comprises computer-readable program code that is configured to establish a plurality of aliases for the single entity in the entity-relationship model that integrates the plurality of databases, a respective alias of which refers to a respective one of the at least two of the identical entities that are identified.

75. A computer program product according to claim 71 further comprising:
computer-readable program code that is configured to traverse the entity-relationship model that integrates the plurality of databases in response to a query to thereby obtain query results that are based on the entity-relationship model that integrates the plurality of databases.

76. A computer program product according to claim 75 wherein the computer-readable program code that is configured to traverse comprises:
computer-readable program code that is configured to traverse the entity-relationship model that integrates the plurality of databases from a starting entity to an ending entity in response to a query that specifies the starting entity and the ending entity to thereby identify relationships between the starting entity and the ending entity that are based on the entity-relationship model that integrates the plurality of databases.

77. A computer program product according to claim 75 wherein the computer-readable program code that is configured to traverse comprises:
computer-readable program code that is configured to traverse the entity-relationship model that integrates the plurality of databases from a starting entity to a plurality of ending entities in response to a query that specifies the starting entity to thereby identify relationships between the starting entity and the plurality of ending entities that are based on the entity-relationship model that integrates the plurality of databases.

78. A computer program product according to claim 75 wherein the computer-readable program code that is configured to traverse comprises:
computer-readable program code that is configured to traverse the entity-relationship model that integrates the plurality of databases in response to a query and in response to at least one path rule to thereby obtain query results that are based on the entity-relationship model that integrates the plurality of databases.

79. A computer program product according to claim 78 wherein the at least one path rule specifies a type of path to use in traversing through the entity-relationship model that integrates the plurality of databases, a type of path not to use in traversing through the entity-relationship model that integrates the plurality of databases, a type of ending entity that can be included in the query results, a type of ending entity that is not to be included in the query results, a type or class of relationship to be used in traversing through the entity-relationship model that integrates the plurality of databases, a type or class of relationship that is not to be used in traversing through the entity-relationship model that integrates the plurality of databases and/or a confidence level to be achieved in traversing through the entity-relationship model that integrates the plurality of databases.

80. A computer program product according to claim 78 further comprising computer-readable program code that is configured to store the query and the path rule for reuse.

81. A computer program product according to claim 75 further comprising:
computer-readable program code that is configured to store the query results that are based on the entity-relationship model that integrates the plurality of databases as at least one new relationship in the entity-relationship model that integrates the plurality of databases to thereby store knowledge that was derived from the query in the entity-relationship model that integrates the plurality of databases.

82. A computer program product according to claim 75 further comprising:
computer-readable program code that is configured to assign a confidence level to at least one of the relationships in the entity-relationship model that integrates the plurality of databases.

83. A computer program product according to claim 82 further comprising:
computer-readable program code that is configured to traverse the entity-relationship model that integrates the plurality of databases in response to a query to thereby obtain query results that are based on the entity-relationship model that integrates the plurality of databases including the at least one confidence level that is assigned.

84. A computer program product that is configured to integrate a new database with a plurality of databases, the computer program product comprising a computer usable storage medium having computer-readable program code embodied in the medium, the computer-readable program code comprising:
an entity-relationship model of the plurality of databases that links at least some related entities in at least two of the databases;
an entity-relationship model of the new database;
computer-readable program code that is configured to identify related entities in the entity-relationship model of the new database and the entity-relationship model of the plurality of databases; and
computer-readable program code that is configured to link at least two of the related entities that are identified, to thereby create an entity-relationship model that integrates the plurality of databases and the new database.

85. A computer program product according to claim 84 wherein the entity-relationship model of the plurality of databases that links at least some related entities in the at least two of the databases provides an ontology network and wherein the entity-relationship model for the new database represents an ontology.

86. A computer program product according to claim 84 wherein the related entities are identical entities and wherein the computer-readable program code that is configured to link comprises computer-readable program code that is configured to merge the at least two of the identical entities that are identified into a single entity in the entity-relationship model that integrates the plurality of databases and the new database.

87. A computer program product according to claim 86 wherein the computer-readable program code that is configured to merge further comprises computer-readable program code that is configured to establish a plurality of aliases for the single entity in the entity-relationship model that integrates the plurality of databases and the new database, a respective alias of which refers to a respective one of the at last two of the identical entities that are identified.

88. A computer program product according to claim 87 wherein the new database is an updated version of one of the plurality of databases, the computer program product further comprising:
computer-readable program code that is configured to identify at least one entity in the one of the plurality of databases that has been deleted from the updated version of the one of the plurality of databases; and
computer-readable program code that is configured to remove an alias that is associated with the at least one entity that has been removed.

89. A computer program product according to claim 88 further comprising:
computer-readable program code that is configured to split at least one entity in the entity-relationship model that integrates the plurality of databases and the new database based upon the alias that was removed.

90. A computer program product according to claim 84 further comprising:
computer-readable program code that is configured to identify entities in the new database that do not correspond to at least one of the entities in the entity-relationship model that integrates the plurality of databases and the new database; and
computer-readable program code that is configured to add at least one new entity to the entity-relationship model that integrates the plurality of databases and the new database that corresponds to the entities in the new database that do not correspond to at least one of the entities in the entity-relationship model that integrates the plurality of databases and the new database.

91. A computer program product according to claim 84 further comprising:
computer-readable program code that is configured to traverse the entity-relationship model that integrates the plurality of databases and the new database in response to a query to thereby obtain query results that are based on the entity-relationship model that integrates the plurality of databases and the new database.

92. A computer program product according to claim 84 further comprising:
computer-readable program code that is configured to traverse the entity-relationship model that integrates the plurality of databases and the new database in response to a query and in response to at least one path rule to thereby obtain query results that are based on the entity-relationship model that integrates the plurality of databases and the new database.

93. A computer program product according to claim 91 further comprising:
computer-readable program code that is configured to store the query results that are based on the entity-relationship model that integrates the plurality of databases and the new database as at least one new relationship in the entity-relationship model that integrates the plurality of databases and the new database to thereby store knowledge that was derived from the query in the entity-relationship model that integrates the plurality of databases and the new database.

94. A computer program products according to claim 84 further comprising:
computer-readable program code that is configured to maintain an image of the entity-relationship model of the plurality of databases before the at least two of the related entities are linked.

95. A computer program product according to claim 94 further comprising:
computer-readable program code that is configured to compare the image of the entity-relationship model of the plurality of databases before the at least two of the related entities are linked and the entity relationship mode that integrates the plurality of biological chemical databases and the new database.

96. A computer program product according to claim 84 wherein the entity-relationship model of the new database does not include relationships therein.

97. A computer program product that is configured to query a plurality of databases, each of which includes records for a plurality of entities, the computer program product comprising a computer usable storage medium having computer-readable program code embodied in the medium, the computer-readable program code comprising:
an integrated entity-relationship model of the plurality of databases that links at least some related entities in at least two of the databases; and
computer-readable program code that is configured to traverse the integrated entity-relationship model of the plurality of databases in response to a query to thereby obtain query results that are based on the integrated entity-relationship model of the plurality of databases.

98. A computer program product according to claim 97 wherein the computer-readable program code that is configured to traverse comprises:
computer-readable program code that is configured to traverse the integrated entity-relationship model of the plurality of databases from a starting entity to an ending entity in response to a query that specifies the starting entity and the ending entity to thereby identify relationships between the starting entity and the ending entity that are based on the integrated entity-relationship model of the plurality of databases.

99. A computer program product according to claim 97 wherein the computer-readable program code that is configured to traverse comprises:
computer-readable program code that is configured to traverse the integrated entity-relationship model of the plurality of databases from a starting entity to a plurality of ending entities in response to a query that specifies the starting entity to thereby identify relationships between the starting entity and the plurality of ending entities that are based on the integrated entity-relationship model of the plurality of databases.

100. A computer program product according to claim 97 wherein the computer-readable program code that is configured to traverse comprises:
computer-readable program code that is configured to traverse the integrated entity-relationship model of the plurality of databases in response to a query and in response to at least one path rule to thereby obtain query results that are based on the integrated entity-relationship model of the plurality of databases.

101. A computer program product according to claim 100 wherein the at least one path rule specifies a type of path to use in traversing through the plurality of entities, a type of path not to use in traversing through the plurality of entities, a type of ending entity that can be included in the query results, a type of ending entity that is not to be included in the query results, a type or class of relationship that is to be used in traversing through the plurality of entities, a type or class of relationship not to be used in traversing through the plurality of entities and/or a confidence level to be achieved in traversing through the plurality of entities.

102. A computer program products according to claim 100 further comprising computer-readable program code that is configured to store the query and the path rule for reuse.

103. A computer program product according to claim 97 further comprising:
computer-readable program code that is configured to store the query results that are based on the integrated entity-relationship model of the plurality of databases as at least one new relationship in the integrated entity-relationship model of the plurality of databases to thereby store knowledge that was derived from the query in the integrated entity-relationship model of the plurality of databases.

104. A computer program product according to claim 97 further comprising:
computer-readable program code that is configured to assign a confidence level to at least one of the relationships in the integrated entity-relationship model of the plurality of databases.

105. A computer program product according to claim 104 further comprising:
computer-readable program code that is configured to traverse the integrated entity-relationship model of the plurality of databases in response to a query to thereby obtain query results that are based on the integrated entity-relationship model of the plurality of databases including the at least one confidence level that is assigned.

106. A data processing system comprising:
an ontology network engine that is configured to build an integrated entity-relationship model of a plurality of independent databases, each of which includes records for a plurality of objects, the integrated entity-relationship model comprising:
a plurality of entities, a respective one of which corresponds to a single object, at least some of the entities including a plurality of links, a respective one of which directly or indirectly refers to at least one record in a respective one of the plurality of databases that relates to the single object; and
a plurality of relationships that link the plurality of entities in the entity-relationship model based upon relationships therebetween.

107. A system according to claim 106 further comprising:
a metadata database that is configured to store therein the integrated entity-relationship model of the plurality of independent databases.

108. A system according to claim 106 further comprising:
a loader that is configured to load an independent entity-relationship model of each of the independent databases into the ontology network engine.

109. A system according to claim 108 wherein the loader is configured to load an independent entity-relationship model of each of the independent databases into the ontology network engine in a typeless format.

110. A system according to claim 108 in combination with the plurality of independent databases.

111. A system according to claim 106 further comprising:
a query tool that is configured to traverse the integrated entity-relationship model in response to a query to thereby obtain query results that are based on the integrated entity-relationship model.

112. A system according to claim 111 wherein the query tool is a Web-based query tool.

113. A system according to claim 106 further comprising:
a virtual experiment tool that is configured to conduct virtual experiments on the integrated entity-relationship model.

114. A system according to claim 106 further comprising:
a discovery tool that is configured to discover knowledge from the integrated entity-relationship model.

115. A system according to claim 106 wherein the ontology network engine runs on a plurality of data processing systems that are configured in a peer-to-peer configuration.

116. A data structure comprising:
an integrated entity-relationship model of a plurality of independent databases, each of which includes records for a plurality of objects, the integrated entity-relationship model comprising:
a plurality of entities, a respective entity of which corresponds to a single object, at least some of the entities including a plurality of links, a respective one of which directly or indirectly refers to at least one record in a respective one of the plurality of databases that relates to the single object; and
a plurality of relationships that link the plurality of entities in the entity-relationship model based upon relationships therebetween.

117. A data structure according to claim 116 further comprising:
an independent entity-relationship model of each of the independent databases.
